(19) United States
(12) Patent Application Publication
(10) Pub. No.: US 2010/0041159 A1
(43) Pub. Date: Feb. 18, 2010

(54) HYDROPHOBIC COMPOUND CAPTURE-APPARATUS MADE FROM BIODEGRADABLE POLYMERS AND METHODS BASED THEREON

(76) Inventors: Christopher T. Nomura, Syracuse, NY (US); Jingnan Lu, Cicero, NY (US)

Correspondence Address: MARJAMA MULDOON BLASIAK & SULLIVAN LLP, 250 SOUTH CLINTON STREET, SUITE 300, SYRACUSE, NY, 13202, US

(21) Appl. No.: 12/503,719

(22) Filed: Jul. 15, 2009

Related U.S. Application Data
(60) Provisional application No. 61/081,181, filed on Jul. 16, 2008.

(51) Int. Cl. B01D 53/02; G01N 33/44; G01N 30/00; G01N 30/02; C12P 7/62

(52) U.S. Cl. 436/85; 95/141; 96/154; 422/88; 422/89; 435/135

(57) ABSTRACT

An apparatus and methods for concentrating or removing hydrophobic organic compounds (HOCs) are provided. A biodegradable polyhydroxyalkanoate (PHA) polymer is provided and the HOC (or a material containing a HOC) is contacted with the PHA. PHA polymers can be used to easily concentrate or remove microcontaminant levels as low as 0.01 ppm of hydrophobic organic compounds without using large solvent volumes. Methods for synthesizing the PHAs are also provided. Methods are further provided for degrading HOCs that have been sequestered from a HOC-contaminated material using isolated bacterial strains or microbes such as Escherichia, Pseudomonas and others. The method comprises contacting at least one isolated bacterial strain (or microbe) to the sequestered HOC for a time sufficient to effect removal of the HOC. The provided methods are advantageous for concentrating HOCs and providing a platform for microbial remediation of these compounds.

### CROSS-REFERENCE TO RELATED APPLICATIONS

This application claims priority to and the benefit of co-pending U.S. provisional patent application Ser. No. 61/081,181 entitled Hydrophobic Compound Capture-Apparatus Made from Biodegradable Polymers, filed Jul. 16, 2008, which is incorporated herein by reference in its entirety.

### STATEMENT REGARDING FEDERALLY SPONSORED RESEARCH OR DEVELOPMENT

Not applicable

### 1. TECHNICAL FIELD

The present invention relates to methods and compounds for concentrating or removing hydrophobic organic compounds (HOCs) from gases, gaseous fluids, fluids or solids.

### 2. BACKGROUND OF THE INVENTION

Polycyclic Aromatic Hydrocarbons (PAHs) represent one of the most widespread organic pollutants and are suspected human carcinogens. Composed of fused aromatic rings, PAHs form primarily through the incomplete combustion of fossil fuels. Removal of these compounds from the environment has been a subject of intense research in recent years. PAHs are hydrophobic and often extremely toxic compounds in aqueous environments (Westerhoff, P., Y. Yoon, S. Snyder, and E. Wert. 2005. Fate of endocrine-disruptor, pharmaceutical, and personal care product chemicals during simulated drinking water treatment processes. Environmental Science & Technology 39:6649-6663). An example of a PAH is naphthalene, a chemical that is used in carbamate insecticides, surface active agents and resins, insect repellents, as a dye intermediate, as a synthetic tanning reagent, and in miscellaneous organic chemicals (Westerhoff, P., Y. Yoon, S. Snyder, and E. Wert. 2005. Fate of endocrine-disruptor, pharmaceutical, and personal care product chemicals during simulated drinking water treatment processes. Environmental Science & Technology 39:6649-6663). Short-term exposure to naphthalene has been associated with hemolytic anemia, liver damage, and neurological damage. Long-term exposure to this toxin has been shown to cause cataracts and damage to the retina (Westerhoff, P., Y. Yoon, S. Snyder, and E. Wert. 2005. Fate of endocrine-disruptor, pharmaceutical, and personal care product chemicals during simulated drinking water treatment processes. Environmental Science & Technology 39:6649-6663). PAHs are also extremely toxic to aquatic organisms.

Current techniques for isolation and analysis of PAHs include the use of liquid-liquid extraction (LLE), accelerated solvent extraction (ASE; also referred to as pressurized fluid extraction), solid phase extraction (SPE) materials such as XAD-2 resin, but may require days of extraction and large volumes of solvent (Olivella, M. A. 2006. Isolation and analysis of polycyclic aromatic hydrocarbons from natural water using accelerated solvent extraction followed by gas chromatography-mass spectrometry. Talanta 69:267-275), semi-permeable membrane devices (SPMD) (Huckins, J. N., J. D. Petty, J. A. Lebo, C. E. Orazio, and D. E. Prest. 1997. Accumulation of organochlorine pesticides and PCBs by semipermeable membrane devices and Mytilus edulis in New Bedford Harbor—Comment. Environmental Science & Technology 31:3732-3733, Huckins, J. N., J. D. Petty, C. E. Orazio, J. A. Lebo, R. C. Clark, V. L. Gibson, W. R. Gala, and K. R. Echols. 1999. Determination of uptake kinetics (Sampling rates) by lipid-containing semipermeable membrane devices (SPMDs) for polycyclic aromatic hydrocarbons (PAHs) in water. Environmental Science & Technology 33:3918-3923, van Stee, L. L. P., P. E. G. Leonards, W. M. G. M. van Loon, A. J. Hendriks, J. L. Maas, J. Struijs, and U. A. T. Brinkman. 2002. Use of semi-permeable membrane devices and solid-phase extraction for the wide-range screening of microcontaminants in surface water by GC-AED/MS. Water Research 36:4455-4470.). Dependent on the end use, all of these techniques have their disadvantages. For example, LLE methods use large amounts of organic solvents that may be harmful to the environment. Most SPE methods also require large volumes of solvent and may not be as sensitive for the detection of PAHs. SPMD devices are passive devices that require the passage of contaminated liquids through the device and may present time constraints when removing the detected compounds.

There is therefore a need in the art for methods of concentration and/or removal of hydrophobic organic compounds. There is further a need in the art for methods of bioremediation of these compounds.

Citation or identification of any reference in Section 2, or in any other section of this application, shall not be considered an admission that such reference is available as prior art to the present invention.

### 3. SUMMARY OF THE INVENTION

Methods for concentrating and/or removing hydrophobic organic compounds (HOCs) are provided. In one embodiment, a method is provided that comprises providing a biodegradable polymer such as a polyhydroxyalkanoate (PHA), and contacting the HOC with the biodegradable polymer. Biodegradable polymers such as PHAs are advantageous in their ability to concentrate HOCs and provide a platform for microbial remediation of these compounds. PHA polymers can be used to easily concentrate or remove microcontaminant levels as low as 0.01 ppm of hydrophobic organic compounds without using large solvent volumes.

A method is also provided for degrading HOCs that have been sequestered from a HOC-contaminated material using isolated bacterial strains. In one embodiment, the method comprises contacting at least one isolated bacterial strain to the sequestered HOC for a time sufficient to effect removal of the HOC.

A method is also provided for adsorbing a hydrophobic organic compound (“HOC”) from a material contaminated with the HOC comprising:
providing a polyhydroxyalkanoate (PHA) polymer; and
contacting the material contaminated with the HOC with the PHA polymer under conditions wherein the HOC binds (or is capable of binding) with the PHA polymer.

A method is also provided for sequestering a HOC from a material contaminated with the HOC comprising:
loading a PHA polymer that binds (or is capable of binding) with at least one HOC onto a support; and
contacting the PHA polymer loaded onto the support to the material contaminated with the HOC.

In another embodiment, the method can further comprise:
maintaining environmental conditions to sustain HOC adsorption until a desired amount of adsorption of the HOC is achieved.

In another embodiment, the method can further comprise:
maintaining environmental conditions to sustain HOC sequestration until a desired amount of sequestration of the HOC is achieved.

In another embodiment, the PHA polymer can be produced by a microbe, such as an isolated bacterial strain.

In another embodiment, the microbe is selected from the group consisting of Escherichia, Pseudomonas, Alcaligenes; Corynebacterium, Acinetobacter, Rhodococcus, Nocardia, Mycobacterium, Bacillus, Flauobacterium, Cunnighamella, Agmenellum, Phanerochaete, Burkholderia, Sphingomonas, Vibrio, Comamonas, and Gordona.

In another embodiment, the PHA polymer is selected from the group consisting of poly(3-hydroxybutyrate), poly-3-alkanoates (PHA or P(3HA)) such as 3-hydroxypropionate, 3-hydroxyhexanoate, 3-hydroxyoctanoate, 3-hydroxydecanoate, 3-hydroxynonoate, 3-hydroxydodecanoate, 3-hydroxytetradecanoate, 3-hydroxyvalerate, 4-hydroxyalkanoates such as 4-hydroxybutyrate and 4-hydroxyvalerate, 5-hydroxyvalerate, 6-hydroxyhexanoate, phenoxyalkanoates, poly-3-hydroxybutyrate-co-3-hydroxyhexanoate, poly-3-hydroxybutyrate-co-3-hydroxyoctanoate, poly-3-hydroxybutyrate-co-3-hydroxydecanoate, poly-4-hydroxybutyrate, poly-4-hydroxybutyrate-co-3-hydroxybutyrate, poly-4-hydroxybutyrate-co-2-hydroxybutyrate, and copolymers and blends thereof.

In another embodiment, the PHA polymer is a bacterial biopolythioester.

In another embodiment, the material contaminated with the HOC is a gas, gaseous fluid, fluid or solid.

In another embodiment, the PHA polymer comprises one or more units of formula 1: —OCR¹R²(CR³R⁴)ₙCO—;

wherein n is an integer and wherein R¹ R² R³ and R⁴ independently are hydrocarbon radicals.

In another embodiment, the PHA polymer comprises 10 and 100,000, units of formula 1. In another embodiment, the PHA polymer comprises 100 and 30,000 units of formula 1. In another embodiment, n is an integer between 1 and 15.

In another embodiment, the hydrocarbon radicals are selected from the group consisting of long chain hydrocarbon radicals, halo- and hydroxy-substituted radicals, hydroxy radicals, halogen radicals, nitrogen-substituted radicals, oxygen-substituted radicals, and/or hydrogen atoms.

In another embodiment, the PHA polymer is a fiber. In another embodiment, the PHA polymer is a film.

A method for remediating a material contaminated with a HOC is also provided. The method can comprise:
providing a PHA polymer;
contacting the material contaminated with the HOC with the PHA polymer under conditions wherein the HOC binds (or is capable of binding) with the PHA to form a PHA polymer-HOC complex; and
removing the HOC from the PHA polymer-HOC complex.

In one embodiment, the material is a gas, gaseous fluid, fluid or solid.

In another embodiment, the method can additionally comprise:
providing a microbe that catabolizes or sequesters (or is capable of catabolizing or sequestering) the PHA polymer,
wherein the step of removing the HOC from the PHA polymer-HOC complex comprises catabolism or sequestration by the microbe.

In another embodiment, the microbe is selected from the group consisting of Escherichia, Pseudomonas, Alcaligenes; Corynebacterium, Acinetobacter, Rhodococcus, Nocardia, Mycobacterium, Bacillus, Flavobacterium, Cunnighamella, Agmenellum, Phanerochaete, Burkholderia, Sphingomonas, Vibrio, Comamonas, and Gordona.

A method for detecting a HOC in a material is also provided. In one embodiment, the method can comprise:
providing a PHA polymer;
contacting the material contaminated with the HOC with the PHA polymer under conditions wherein the HOC is binds (or is capable of binding) with the PHA polymer; and
assaying for the presence of HOC bound with the PHA polymer.

In one embodiment, the material is a gas, gaseous fluid, fluid or solid.

In another embodiment, the assaying step can comprise determining a concentration of the HOC bound with the PHA polymer.

In another embodiment, the assaying step can comprise performing gas chromatography and mass spectroscopy (GC/MS). In another embodiment, the assaying step can comprise performing NMR.

An apparatus for adsorbing a HOC from a material contaminated with the HOC is also provided. The apparatus can comprise a PHA polymer that binds (or is capable of binding) with at least one HOC, wherein the PHA polymer is disposed in the apparatus in an orientation so that it contacts (or is capable of contacting) the material contaminated with the HOC.

In one embodiment, the PHA polymer can be loaded on a support. In another embodiment, the PHA polymer is a fiber. In another embodiment, the PHA polymer is a film.

In another embodiment, the apparatus maintains (or is capable of maintaining) environmental conditions to sustain HOC adsorption or sequestration until a desired amount of adsorption or sequestration of the HOC is achieved.

In another embodiment, the apparatus can be a filter, wand, cone or tube. In another embodiment, the apparatus can be a coating.

In another embodiment, the support is selected from the group consisting of metal rod, metal tube, metal mesh, glass rod, glass tube or glass mesh. In another embodiment, the support can be a gelatinous medium. In another embodiment, the support is selected from the group consisting of gels, pastes, paper, and aqueous solutions.

In another embodiment, the support can be a filter, such as filter paper or a filter made from a synthetic material. Many such synthetic materials are commonly known in the art as suitable for filters.

An apparatus for detecting a HOC in a material is also provided. The apparatus can comprise a PHA polymer that binds (or is capable of binding) with at least one HOC, wherein:
the PHA polymer is disposed in the apparatus in an orientation so that it contacts (or is capable of contacting) the material;
the PHA polymer binds (or is capable of binding) with the HOC in the material; and the presence of the HOC in the PHA polymer is determined by performing an assay for the HOC.

In one embodiment, the assay comprises determining a concentration of the HOC bound with the PHA polymer.

In another embodiment, the assay comprises performing gas chromatography and mass spectroscopy (GC/MS).

A method for producing a PHA is also provided. In one embodiment, the method is selected from the group consisting of the synthetic reaction pathways set forth in FIG. 18, reactions #18-1 to #18-15, and combinations (including combinations of sequential reaction pathways) thereof. FIG. 18 is a schematic illustration of a family of synthetic reaction pathways from which PHA intermediate products and PHA final products can be produced. The starting material (precursor PHA) is shown at the top of the figure, from which the synthetic reaction pathway arrows emanate. The steps of the methods for producing various intermediate and final PHA products, with details of the synthetic pathways, are illustrated in FIGS. 19-26. Any of the intermediate or final PHA products can be used in the apparatus, detection and remediation methods described herein.

In another embodiment, the method is selected from the group consisting of the synthetic reaction pathways set forth in FIG. 27, reactions #27-1 to #27-6, and combinations (including combinations of sequential reaction pathways) thereof. FIG. 27 is a schematic illustration of another family of synthetic reaction pathways from which PHA intermediate products and PHA final products can be produced. The starting material (precursor PHA) is shown at the top of the figure, from which the synthetic reaction pathway arrows emanate. The steps of the methods for producing various intermediate and final PHA products, with details of the synthetic pathways, are illustrated in FIGS. 28-38. Any of the intermediate or final PHA products can be used in the apparatus, detection and remediation methods described herein.

### 4. BRIEF DESCRIPTION OF THE DRAWINGS

The present invention is described herein with reference to the accompanying drawings, in which similar reference characters denote similar elements throughout the several views. It is to be understood that in some instances, various aspects of the invention may be shown exaggerated or enlarged to facilitate an understanding of the invention.

Common abbreviations for organic substructures:

| | | |
|---|---|---|
| Me | methyl | CH₃— |
| Et | ethyl | CH₃CH₂— |
| Bu, n-Bu | butyl | CH₃CH₂CH₂CH₂— |
| Ph | phenyl | C₆H₅— |
| Ar | aryl | functional group or substituent derived from simple |
| | | aromatic ring |

FIG. 1 shows poly(3-Hydroxyalkanoates) PHA polymers accumulated by E. coli that were used for the experiment in Section 6.1, Example 1. Two types were used for the experimental data shown. A. PHA fibers. B. PHA film. ˜30 mg of polymer were used for each experiment.

FIGS. 2-8 show detection and removal of various concentrations (0.01-20 ppm) of the HOC naphthalene from aqueous solution using PHA polymer fibers as detected by GC/MS. In each figure, the top chromatograph shows the amount of naphthalene extracted without polymer treatment. The middle chromatograph shows the amount of naphthalene left in solution after polymer treatment. The bottom chromatograph shows the amount of naphthalene extracted from the polymer. Each of these results is divided into two chromatographs with the top chromatograph showing the naphthalene concentration, retention time, and m/z ratio identifying the compound as naphthalene. The second chromatograph shows the anthracene internal control. See Section 6.1 for details.

FIGS. 2A-C show detection and removal of 0.01 ppm naphthalene from aqueous solution using PHA polymer fibers as detected by GC/MS. A. Pre-polymer treatment. B. Post-polymer treatment (30 mg polymer fiber). C. Recovered from polymer. See Section 6.1 for details.

FIGS. 3A-C show detection and removal of 0.1 ppm naphthalene from aqueous solution using PHA polymer fibers as detected by GC/MS. A. Pre-polymer treatment. B. Post-polymer treatment (30 mg polymer fiber). C. Recovered from polymer. See Section 6.1 for details.

FIGS. 4A-C show detection and removal of 1 ppm naphthalene from aqueous solution using PHA polymer fibers as detected by GC/MS. A. Pre-polymer treatment. B. Post-polymer treatment (30 mg polymer fiber). C. Recovered from polymer. See Section 6.1 for details.

FIGS. 5A-C show detection and removal of 2 ppm naphthalene from aqueous solution using PHA polymer fibers as detected by GC/MS. A. Pre-polymer treatment. B. Post-polymer treatment (30 mg polymer fiber). C. Recovered from polymer. See Section 6.1 for details.

FIGS. 6A-C show detection and removal of 5 ppm naphthalene from aqueous solution using PHA polymer fibers as detected by GC/MS. A. Pre-polymer treatment. B. Post-polymer treatment (30 mg polymer fiber). C. Recovered from polymer. See Section 6.1 for details.

FIGS. 7A-C shows detection and removal of 10 ppm naphthalene from aqueous solution using PHA polymer fibers as detected by GC/MS. A. Pre-polymer treatment. B. Post-polymer treatment (30 mg polymer fiber). C. Recovered from polymer. See Section 6.1 for details.

FIGS. 8A-C shows detection and removal of 20 ppm naphthalene from aqueous solution using PHA polymer fibers as detected by GC/MS. A. Pre-polymer treatment. B. Post-polymer treatment (30 mg polymer fiber). C. Recovered from polymer. See Section 6.1 for details.

FIG. 9 shows naphthalene treatment curve for PHA polymer fibers. The peak area ratio of naphthalene area to anthracene area is plotted against naphthalene concentration (ppm). See Section 6.1 for details.

FIGS. 10-16 show detection and removal of various concentrations (0.01-20 ppm) of naphthalene from aqueous solution using PHA polymer film disks as detected by GC/MS. In each figure, the top chromatograph shows the amount of naphthalene extracted without polymer treatment. The middle chromatograph shows the amount of naphthalene left in solution after polymer treatment. The bottom chromatograph shows the amount of naphthalene extracted from the polymer. Each of these results is divided into two chromatographs with the top chromatograph showing the naphthalene concentration, retention time, and m/z ratio identifying the compound as naphthalene. The second chromatograph shows the anthracene internal control. See Section 6.1 for details.

FIGS. 10A-C show detection and removal of 0.01 ppm naphthalene from aqueous solution using PHA polymer film disks as detected by GC/MS. A. Pre-polymer treatment. B. Post-polymer treatment (30 mg polymer film/disk). C. Recovered from polymer. See Section 6.1 for details.

FIGS. 11A-C show detection and removal of 0.1 ppm naphthalene from aqueous solution using PHA polymer film disks as detected by GC/MS. A. Pre-polymer treatment. B. Post-polymer treatment (30 mg polymer film/disk). C. Recovered from polymer. See Section 6.1 for details.

FIGS. 12A-C show detection and removal of 1 ppm naphthalene from aqueous solution using PHA polymer film disks as detected by GC/MS. A. Pre-polymer treatment. B. Post-polymer treatment (30 mg polymer film/disk). C. Recovered from polymer. See Section 6.1 for details.

FIGS. 13A-C show detection and removal of 2 ppm naphthalene from aqueous solution using PHA polymer film disks as detected by GC/MS. A. Pre-polymer treatment. B. Post-polymer treatment (30 mg polymer film/disk). C. Recovered from polymer. See Section 6.1 for details.

FIGS. 14A-C show detection and removal of 5 ppm naphthalene from aqueous solution using PHA polymer film disks as detected by GC/MS. A. Pre-polymer treatment. B. Post-polymer treatment (30 mg polymer film/disk). C. Recovered from polymer. See Section 6.1 for details.

FIGS. 15A-C shows detection and removal of 10 ppm naphthalene from aqueous solution using PHA polymer film disks as detected by GC/MS. A. Pre-polymer treatment. B. Post-polymer treatment (30 mg polymer film/disk). C. Recovered from polymer. See Section 6.1 for details.

FIGS. 16A-C show detection and removal of 20 ppm naphthalene from aqueous solution using PHA polymer film disks as detected by GC/MS. A. Pre-polymer treatment. B. Post-polymer treatment (30 mg polymer film/disk). C. Recovered from polymer. See Section 6.1 for details.

FIG. 17 shows naphthalene treatment curve for PHA polymer disks. The peak area ratio of naphthalene area to anthracene area (y-axis) is plotted against naphthalene concentration in ppm (x-axis). See Section 6.1 for details.

FIG. 18 is a schematic illustration of a family of synthetic reaction pathways from which PHA intermediate products and PHA final products can be produced. See Section 5 (“Synthesis of PHA monomers and polymers”) for detail.

FIGS. 19-26 show enlarged details of the steps of the synthetic reaction pathways illustrated in FIG. 18.

FIG. 27 is a schematic illustration of another family of synthetic reaction pathways from which PHA intermediate products and PHA final products can be produced. See Section 5 (“Synthesis of PHA monomers and polymers”) for detail.

FIGS. 28-38 show enlarged details of the steps of the synthetic reaction pathways illustrated in FIG. 27.

FIG. 39 illustrates the steps of one embodiment of a method for PAH bioremediation using PHA. See Section 6.2 for details.

FIG. 40 shows the extraction procedure used for the removal of PAHs from an aqueous sample as described in Section 6.2. PHA (30 mg) fiber (A) or film (B) was added to each of the 10 ml water with naphthalene concentration of 0.01 ppm, 0.1 ppm, 1 ppm, 2 ppm, 5 ppm, 10 ppm, and 20 ppm or phenanthrene concentration of 0.001 ppm, 0.01 ppm, 0.125 ppm, 0.25 ppm, 0.5 ppm, and 1 ppm (1). After reaching equilibrium (6 h), PHA polymer-naphthalene complex were taken out and extracted with 2 mL hexanes in another vial to desorb PAH from PHA (2). The remaining aqueous solution after PHA treatment was also extracted with hexanes for comparison with the polymer extricant (3).

FIG. 41 shows the P[3HB] fibers and film accumulated by E. coli as described in Section 6.2.

FIG. 42 shows phenanthrene (0.2 ppm) extraction using hexanes as described in Section 6.2. Extraction times tested were 1 min, 5 min, 15 min, 20 min, 60 min, and 720 min.

FIG. 43 shows naphthalene sequestration by P[3HB] polymer fiber as described in Section 6.2. The naphthalene concentrations tested were 0.01 ppm, 0.1 ppm, 1 ppm, 2 ppm, 5 ppm, 10 ppm, and 20 ppm. The amount of naphthalene removed was 100%, 95%, 95%, 96%, 96%, 95%, and 94% respectively. Left bar graph of each pair at each concentration represents pre-treatment; right bar graph represents post-treatment.

FIG. 44 shows naphthalene removal by P[3HB] polymer fiber as described in Section 6.2. Diamonds, pretreatment. Triangles, recovery. Squares, post-treatment.

FIG. 45 shows naphthalene sequestration by P[3HB] polymer films as described in Section 6.2. Naphthalene concentrations tested were 0.01 ppm, 0.1 ppm, 1 ppm, 2 ppm, 5 ppm, and 20 ppm. The amount of naphthalene removed was 100%, 81%, 88%, 86%, 95%, and 86% respectively. Left bar graph of each pair at each concentration represents pre-treatment; left bar graph represents post-treatment.

FIG. 46 shows naphthalene removal by P[3HB] polymer films as described in Section 6.2. Diamonds, pretreatment. Triangles, recovery. Squares, post-treatment.

FIG. 47 shows phenanthrene sequestration by P[3HB] polymer fibers as described in Section 6.2. The phenanthrene concentrations tested were 0.001 ppm, 0.01 ppm, 0.125 ppm, 0.25 ppm, 0.5 ppm, and 1 ppm. The amount of phenanthrene removed was 65%, 94%, 96%, 95%, 94%, and 96% respectively. Left bar graph of each pair at each concentration represents pre-treatment; left bar graph represents post-treatment.

FIG. 48 shows phenanthrene removal by P[3HB] polymer fibers as described in Section 6.2. Diamonds, pretreatment. Triangles, recovery. Squares, post-treatment.

FIG. 49 shows phenanthrene sequestration by P[3HB] polymer fibers in the presence of humic substances as described in Section 6.3. Phenanthrene concentrations of 0.01 ppm, 0.1 ppm, 0.25 ppm, 0.5 ppm, and 1 ppm were tested. The amount of phenanthrene removed was 99%, 100%, 100%, 100%, and 99% respectively. Left bar graph of each pair at each concentration represents pre-treatment; left bar graph represents post-treatment.

FIG. 50 shows phenanthrene removal by P[3HB] polymer fiber in the presence of humic substances as described in Section 6.3. Diamonds, pretreatment. Triangles, recovery. Squares, post-treatment.

### 5. DETAILED DESCRIPTION OF THE INVENTION

Methods and apparatuses for adsorbing hydrophobic organic compounds (HOCs), including but not limited to polyaromatic hydrocarbons (PAHs) and polychlorobiphenyls (PCBs), are provided. In one embodiment, the method comprises contacting a biodegradable polymer from the PHA class of biodegradable polymers with the HOC. The method can be used, for example, as a detection probe and for bioremediation.

The method can be used to adsorb HOCs from gases, gaseous fluids, fluids or solids.

The term “fluid,” as used herein, refers to any fluid, whether in liquid or gaseous form, or more generally, any substance that deforms continuously under the action of an applied shear force or stress.

In another embodiment, a method for sequestering a HOC from a material contaminated with the HOC is provided that employs microbial catabolism. The method can be used, for example, for the sequestration, removal and or bioremediation of hydrophobic contaminants in an aqueous environment.

In one embodiment, the method can comprise:
loading a PHA polymer that binds (or is capable of binding) with at least one HOC onto a support; and
contacting the PHA polymer loaded onto the support to the material contaminated with the HOC.

In another embodiment, the method can further comprise:
maintaining environmental conditions to sustain HOC adsorption until a desired amount of adsorption of the HOC is achieved.

In another embodiment, the method can further comprise:
maintaining environmental conditions to sustain HOC sequestration until a desired amount of sequestration of the HOC is achieved.

In another embodiment, the PHA polymer can be produced by a microbe, such as an isolated bacterial strain.

In another embodiment, a method and an apparatus for detection of microcontaminant levels of hydrophobic contaminants in the environment are provided.

A method is also provided for detecting a HOC in a material comprising:
providing a PHA polymer;
contacting the material with the PHA polymer under conditions wherein the HOC binds (or is capable of binding) with the PHA polymer; and
assaying for the presence of HOC bound with the PHA polymer.

In one embodiment, the assaying step can comprise determining a concentration of the HOC bound with the PHA polymer.

In another embodiment, the assaying step can comprise performing gas chromatography and mass spectroscopy (GC/MS). In another embodiment, the assaying step can comprise performing high performance liquid chromatography (HPLC), HPLC/MS, GC or NMR.

In one embodiment, the apparatus can comprise a PHA polymer that binds (or is capable of binding) with at least one HOC, wherein:
the PHA polymer is disposed in the apparatus in an orientation so that it is capable of contacting the material;
the PHA polymer binds (or is capable of binding) with the HOC; and
the PHA polymer is capable of being assayed for the presence of the HOC.

In another embodiment, the apparatus can be a filter apparatus for assaying for, or filtering of, gaseous hydrophobic organic contaminants.

In another embodiment, the apparatus can be a filter apparatus for assaying for, or filtering of, hydrophobic organic contaminants from aqueous environments or systems.

In another embodiment, a carbon source for the production of new PHAs is provided. PH As can be derived from any precursor known in the art, such as fatty acids, sugars, alcohols, alkanes, alkenes, carbon dioxide and organic acids. These carbon sources have been described in detail in the literature (Keenan, T. M., J. P. Nakas, and S. W. Tannenbaum. 2006. Polyhydroxyalkanoate copolymers from forest biomass. J Ind Microbiol Biotechnol 33:616-626; O'Leary, N. D., K. E. O'Connor, P. Ward, M. Goff, and A. D. Dobson. 2005. Genetic characterization of accumulation of polyhydroxyalkanoate from styrene in Pseudomonas putida CA-3. Appl Environ Microbiol 71:4380-7; Steinbuchel, A., and B. Fuchtenbusch. 1998. Bacterial and other biological systems for polyester production. Trends Biotechnol 16:419-27; Sudesh, K. 2004. Microbial polyhydroxyalkanoates (PHAs): an emerging biomaterial for tissue engineering and therapeutic applications. Med J Malaysia 59 Suppl B:55-6; and references therein).

For clarity of disclosure, and not by way of limitation, the detailed description of the invention is divided into the subsections set forth below.

### 5.1 Polyhydroxyalkanoates (PHAs)

Polyhydroxyalkanoates (PHAs) are bacterially produced, biobased, and biodegradable alternatives to petroleum-based plastics that could alleviate many of the problems associated with petroleum-based plastics (Anderson, A. J., and E. A. Dawes. 1990. Occurrence, metabolism, metabolic role, and industrial uses of bacterial polyhydroxyalkanoates. Microbiol Rev 54:450-72; Sudesh, K., H. Abe, and Y. Doi. 2000. Synthesis, structure and properties of polyhydroxyalkanoates: biological polyesters. Prog. Polym. Sci. 25:1503-1555). PHAs are ‘green’ biopolyesters that can be produced by bacteria from renewable feedstocks, and unlike petroleum based plastics, can be completely mineralized to CO₂ and H₂O upon disposal or can be easily recycled (Anderson, A. J., and E. A. Dawes. 1990. Occurrence, metabolism, metabolic role, and industrial uses of bacterial polyhydroxyalkanoates. Microbiol Rev 54:450-72; Sudesh, K., H. Abe, and Y. Doi. 2000. Synthesis, structure and properties of polyhydroxyalkanoates: biological polyesters. Prog. Polym. Sci. 25:1503-1555). These polymers have a number of uses: as bulk-commodity plastics, in marine environments, and in a variety of high-value medical applications (Anderson, A. J., and E. A. Dawes. 1990. Occurrence, metabolism, metabolic role, and industrial uses of bacterial polyhydroxyalkanoates. Microbiol Rev 54:450-72; Sudesh, K., H. Abe, and Y. Doi. 2000. Synthesis, structure and properties of polyhydroxyalkanoates: biological polyesters. Prog. Polym. Sci. 25:1503-1555). PHAs have physical properties based on the number of carbon atoms in the individual monomer units incorporated by bacterial enzymes into the polymer chains. Short-chain-length (scl) PHAs consist of monomers of C3-C5 in length and medium-chain-length (mcl) PHA consists of monomers of C6-C14 in length. Polymers composed of scl have thermoplastic properties, while polymers composed of mcl subunits have elastomeric properties.

According to the invention, PHA polymers can be employed to efficiently absorb HOCs, providing an effective method of removal from the environment. The hydrophobic nature of PHA polymers causes them to adsorb HOCs from aqueous solutions and colloidal and other organic materials. However, unlike petroleum-based plastics, PHA polymers can act both as an apparatus to capture and concentrate HOC pollutants from aqueous environments and as a vector for the bioremediation of these pollutants, since certain bacteria can metabolize both the polymers (Jendrossek, D., A. Frisse, A. Behrends, M. Andermann, H. D. Kiatzin, T. Stanislawski, and H. G. Schlegel. 1995. Biochemical and molecular characterization of the Pseudomonas lemoignei polyhydroxyalkanoate depolymerase system. J Bacteriol 177:596-607; Jendrossek, D., and R. Handrick. 2002. Microbial degradation of polyhydroxyalkanoates. Annu Rev Microbiol 56:403-32) and the hydrophobic pollutants (Ahn, I. S., W. C. Ghiorse, L. W. Lion, and M. L. Shuler. 1998. Growth kinetics of Pseudomonas putida G7 on naphthalene and occurrence of naphthalene toxicity during nutrient deprivation. Biotechnology and Bioengineering 59:587-594) after such biocontainment applications. The present methods for bioremediation and the present probe/capture apparatus provided by the invention can be used for treating HOC pollutants from aqueous environments.

### 5.2 Hydrophobic Organic Compounds (HOCs)

The methods provided herein are suitable for the sequestration, removal and/or remediation of a wide range of hydrophobic organic compounds (HOCs), including but not limited to polyaromatic hydrocarbons (PAHs) and polychlorobiphenyls (PCBs) and are provided. PAHs include, but are not limited to, naphthalene, fluorene, phenanthrene, n-hexadecane, hexane, benzene, toluene, dinitrotoluene (DNT) and the like. PAH contaminants are well known in the art (see, e.g., Westerhoff, P., Y. Yoon, S. Snyder, and E. Wert (2005. Fate of endocrine-disruptor, pharmaceutical, and personal care product chemicals during simulated drinking water treatment processes. Environmental Science & Technology 39:6649-6663).

PCBs are also well known in the art and include, but are not limited to, 2-chlorobiphenyl, 3-chlorobiphenyl and the like, polybromobiphenyls (PBBs), and diphenylphenols.

In another embodiment, the HOC is substantially non-vaporized. In one embodiment, the HOC can have a molecular formula of up to about C₂₀Hₓ, wherein X varies depending on a level of saturation of the HOC.

In another embodiment, the solubility of the HOC is at least about 1.8 μg/L at room temperature.

### 5.3 Synthesis of PHA Monomers and Polymers

Many PHA polymers suitable for use in the methods of the invention are known in the art. For example, the PHA polymer can be selected from the group consisting of poly(3-hydroxybutyrate), poly-3-alkanoates (PHA or P(3HA)) such as 3-hydroxypropionate, 3-hydroxyhexanoate, 3-hydroxyoctanoate, 3-hydroxydecanoate, 3-hydroxynonoate, 3-hydroxydodecanoate, 3-hydroxytetradecanoate, 3-hydroxyvalerate, 4-hydroxyalkanoates such as 4-hydroxybutyrate and 4-hydroxyvalerate, 5-hydroxyvalerate, 6-hydroxyhexanoate, phenoxyalkanoates, poly-3-hydroxybutyrate-co-3-hydroxyhexanoate, poly-3-hydroxybutyrate-co-3-hydroxyoctanoate, poly-3-hydroxybutyrate-co-3-hydroxydecanoate, poly-4-hydroxybutyrate, poly-4-hydroxybutyrate-co-3-hydroxybutyrate, poly-4-hydroxybutyrate-co-2-hydroxybutyrate, and copolymers and blends thereof.

PHA polymers comprise one or more units, for example between 10 and 100,000, and preferably between 100 and 30,000 units of the following formula I: —OCR¹R²(CR³R⁴)ₙCO—; wherein n is an integer, for example between 1 and 15, and in a preferred embodiment, between 1 and 4; and wherein R^{1,} R^{2,} R³ and R⁴ independently can be hydrocarbon radicals including long chain hydrocarbon radicals; halo- and hydroxy-substituted radicals; hydroxy radicals; halogen radicals; nitrogen-substituted radicals; oxygen-substituted radicals; and/or hydrogen atoms.

As used herein, the formula —(CR³R⁴)ₙ— is defined as including the following formulas: —CR³R⁴— (where n=1); —CR³R⁴CR³′R⁴′— (where n=2); and —CR³R⁴CR³′R⁴′CR³″R⁴″— (where n=3); wherein R³, R⁴, R³′, R⁴′, R³″, and R⁴″ can be independently hydrocarbon radicals including long chain hydrocarbon radicals; halo- and hydroxy-substituted radicals; hydroxy radicals, halogen radicals; nitrogen-substituted radicals; oxygen-substituted radicals; and/or hydrogen atoms. Thus, formula I includes units derived from 3-hydroxyacids (n=1), 4-hydroxyacids (n=2), and 5-hydroxyacids (n=3). These units may be the same in a homopolymer, or be more different units, as for example in a copolymer or terpolymer. The polymers typically have a molecular weight (MW) over 300, for example between 300 and 10⁷ Daltons. In one embodiment, the polymers have a MW of 1000 to greater than 1,000,000. Preferably, the MW range is 400,000-1,000,000.

The PHA polymers may comprise or be modified to include other molecules, such as bioactive and detectable compounds, surface active agents, other degradable or non-degradable polymers, as well as materials used to modify the mechanical properties of PHAs such as plasticizers, filters, nucleating agents, colorants, stabilizers, modifiers and binders.

Representative PHAs that can be modified or formulated for use in the methods and apparatus described herein are described in U.S. Pat. No. 7,268,205, Medical devices and applications of polyhydroxyalkanoate polymers, Williams et al., Sep. 11, 2007; and Steinbuchel & Valentin, FEMS Microbiol. Lett., 128:219-28 (1995).

In another embodiment, the PHA polymers can be bacterial biopolythioesters (containing S atoms) as described by Lutke-Eversloh et al. (Lutke-Eversloh, T., K. Bergander, H. Luftmann, and A. Steinbuchel. 2001. Biosynthesis of poly(3-hydroxybutyrate-co-3-mercaptobutyrate) as a sulfur analogue to poly(3-hydroxybutyrate) (PHB). Biomacromolecules 2:1061-5; Lutke-Eversloh, T., K. Bergander, H. Luftmann, and A. Steinbuchel. 2001. Identification of a new class of biopolymer: bacterial synthesis of a sulfur-containing polymer with thioester linkages. Microbiology 147:11-9; and Lutke-Eversloh, T., J. Kawada, R. H. Marchessault, and A. Steinbuchel. 2002. Characterization of microbial polythioesters: physical properties of novel copolymers synthesized by Ralstonia eutropha. Biomacromolecules 3:159-66).

New PHA monomer structures can be designed and synthesized using organic synthesis methods well known in the art (see, e.g., U.S. Pat. No. 7,208,535, PHA compositions and methods for their use in the production of PHA films, Asrar et al., issued Apr. 24, 2007; Grossman, R. B. 2003, The Art of Writing Reasonable Organic Reaction Mechanisms, vol. Springer, New York).

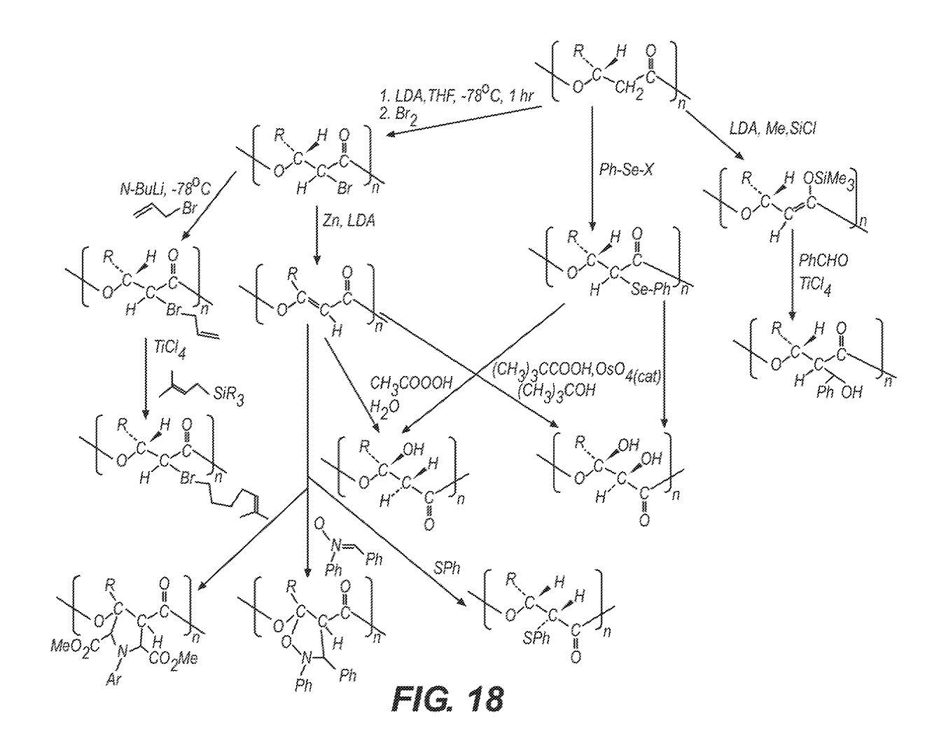

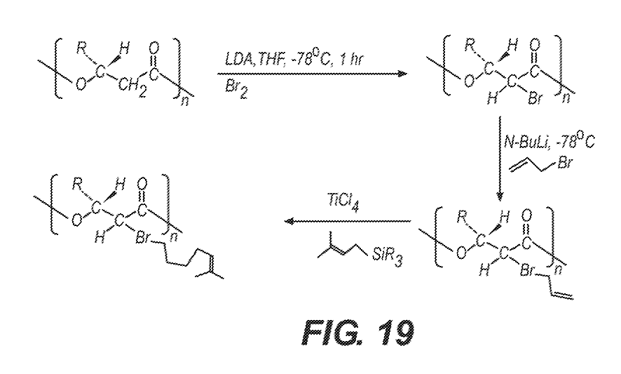

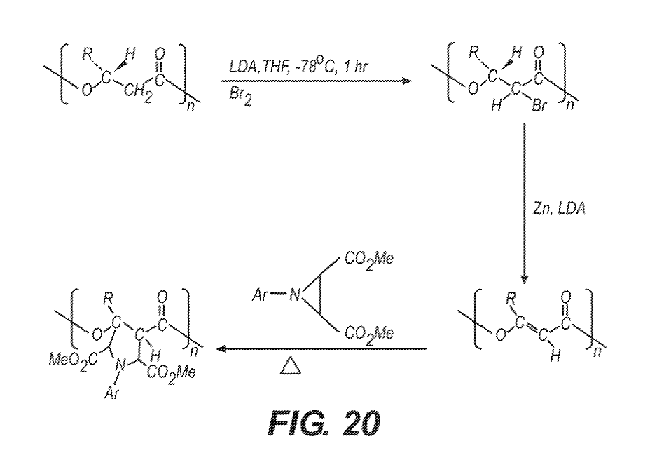

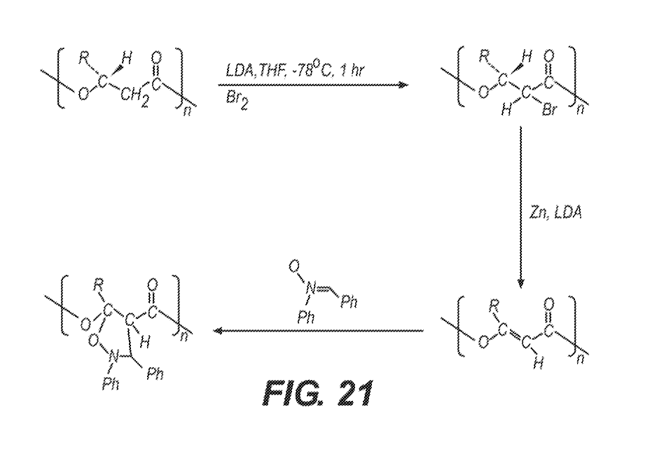

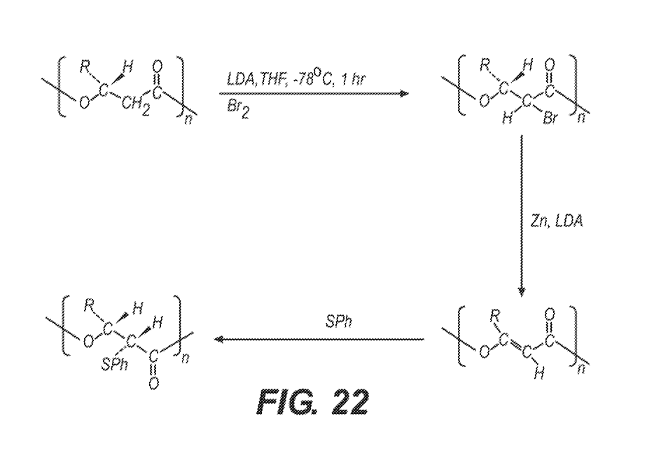

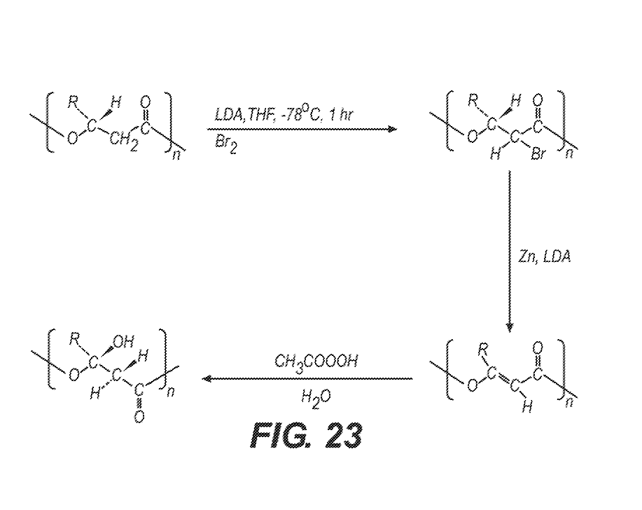

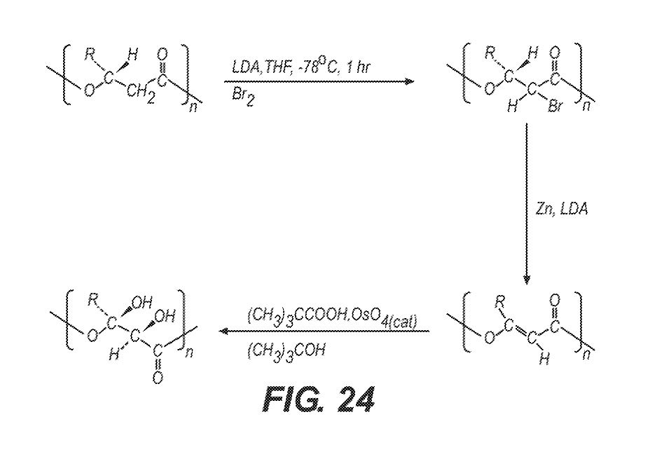

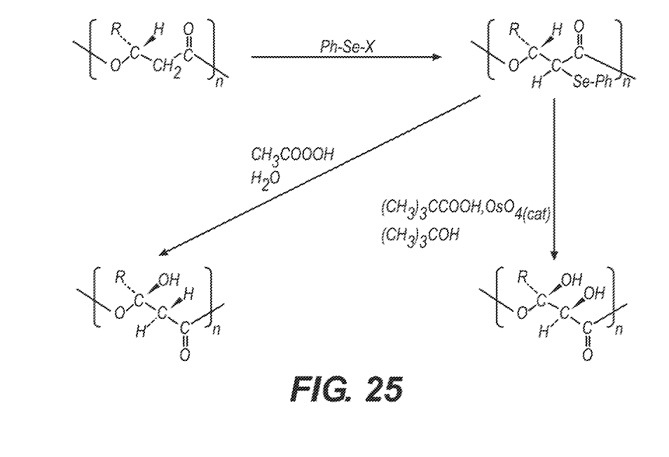

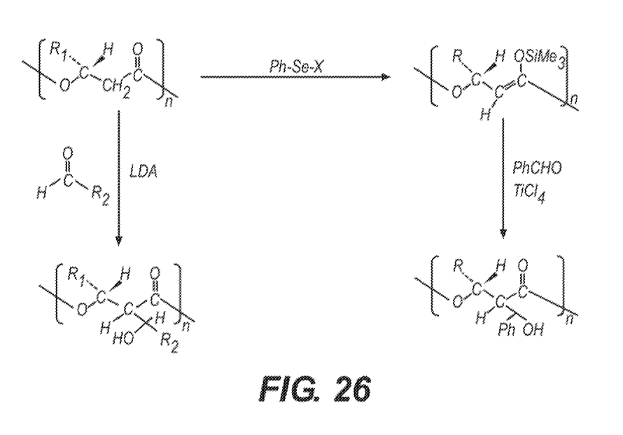

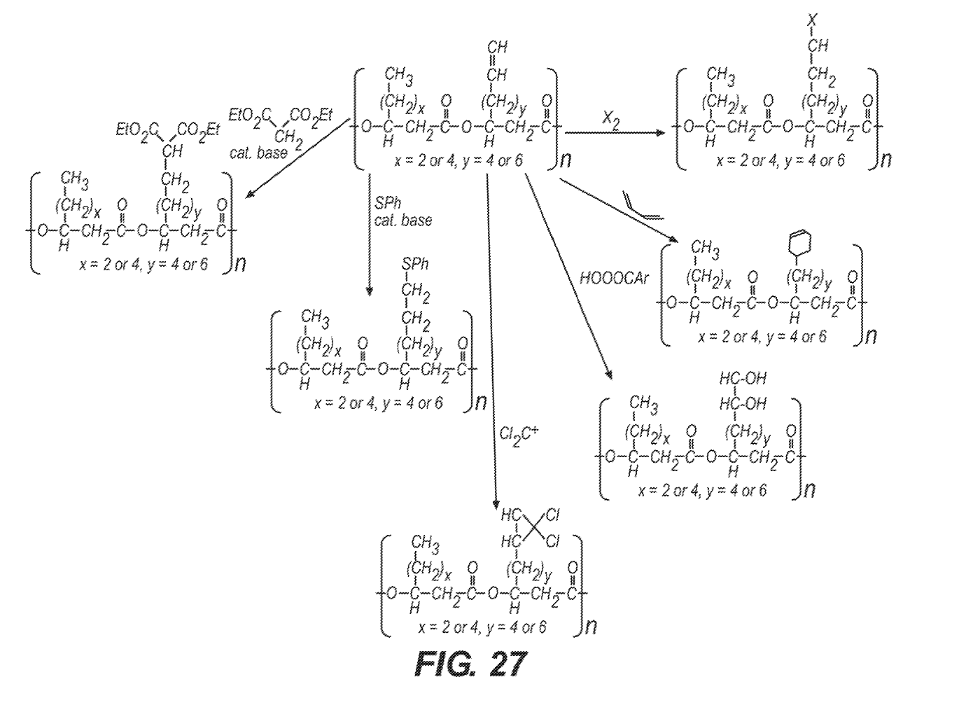

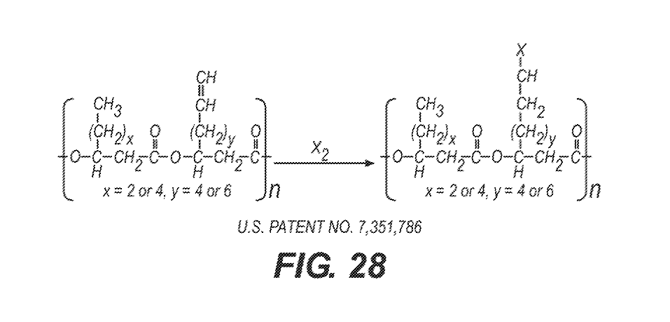

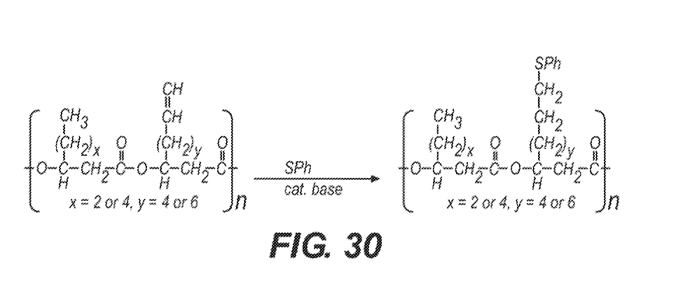

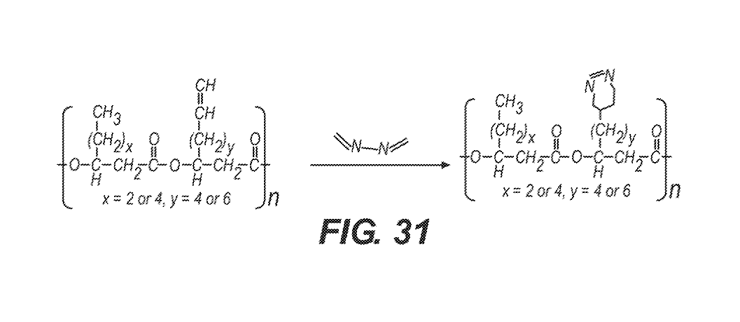

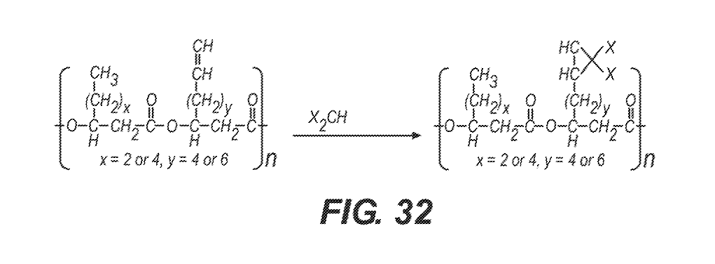

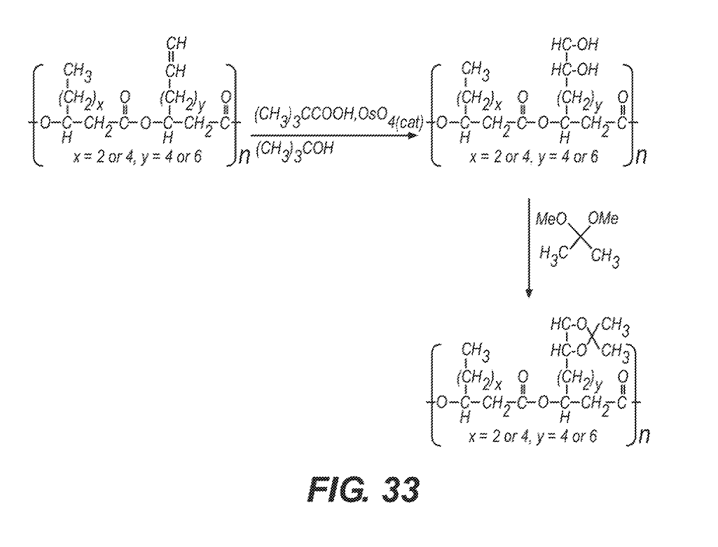

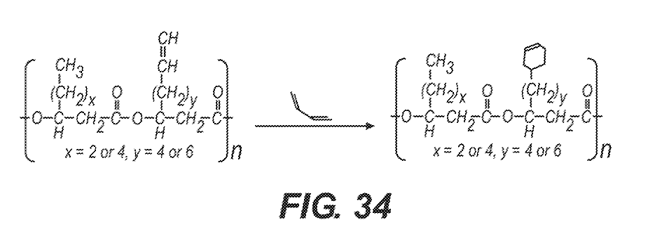

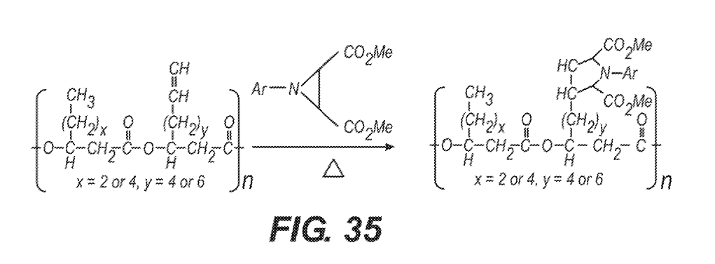

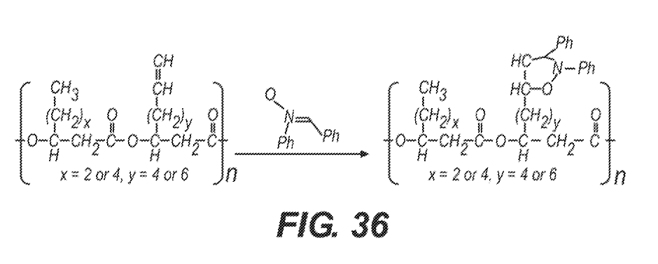

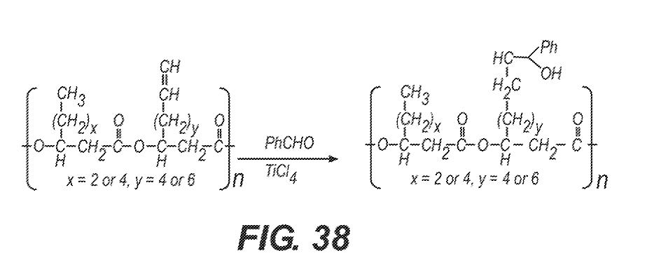

A method for synthesizing new PHA monomer structures is also provided. In one embodiment, the method can comprise a synthetic reaction pathway selected from the group consisting of the synthetic reaction pathways illustrated in FIGS. 18-26 or in FIGS. 27-38. The synthetic reaction pathways illustrated in FIG. 18 (with details the pathway illustrated in FIGS. 19-26) and in FIG. 27 (with details of the pathway illustrated in FIGS. 28-38) are pathways that can be used to produce other polymer types starting from precursor PHAs. Such other polymer types can have additional side groups and/or side chains, as is well known in the art.

A method for producing a PHA is also provided. In one embodiment, the method is selected from the group consisting of the synthetic reaction pathways set forth in FIG. 18, reactions #18-1 to #18-15, and combinations (including combinations of sequential reaction pathways) thereof. FIG. 18 is a schematic illustration of a family of synthetic reaction pathways from which PHA intermediate products and PHA final products can be produced. The starting material (precursor PHA) is shown at the top of the figure, from which the synthetic reaction pathway arrows emanate. The steps of the methods for producing various intermediate and final PHA products, with details of the synthetic pathways, are illustrated in FIGS. 19-26. Any of the intermediate or final PHA products can be used in the apparatus, detection and remediation methods described herein.

In another embodiment, the method is selected from the group consisting of the synthetic reaction pathways set forth in FIG. 27, reactions #27-1 to #27-6, and combinations (including combinations of sequential reaction pathways) thereof. FIG. 27 is a schematic illustration of another family of synthetic reaction pathways from which PHA intermediate products and PHA final products can be produced. The starting material (precursor PHA) is shown at the top of the figure, from which the synthetic reaction pathway arrows emanate. The steps of the methods for producing various intermediate and final PHA products, with details of the synthetic pathways, are illustrated in FIGS. 28-38. Any of the intermediate or final PHA products can be used in the apparatus, detection and remediation methods described herein.

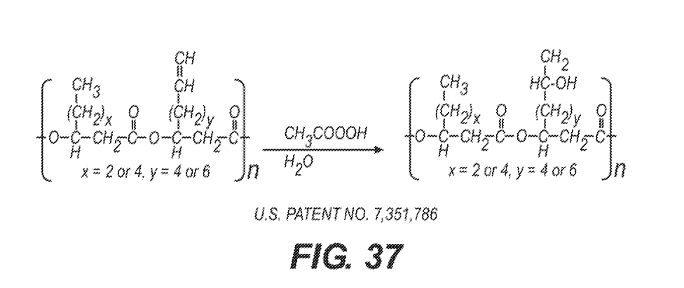

FIGS. 28 and 37 show reactions that can be used according to these methods and that are disclosed in U.S. Pat. No. 7,351,786 (Honma et al., issued Apr. 1, 2008, entitled Polyhydroxyalkanoate containing unit having cyclohexyl structure at side chain, production process therefor, and binder resin containing polyhydroxyalkanoate).

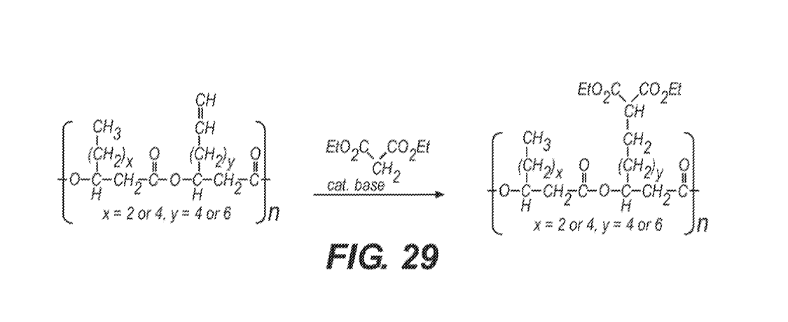

In a specific embodiment, a method is provided for synthesizing a PHA as shown in FIGS. 29-36 and 38 (as well as in the overview schematic of FIG. 27).

The PHA can be biologically produced, e.g., by a plant or microbial organism. For example, it can be a fermentation product, particularly of a microbiological process, whereby a microorganism lays down PHA during normal or manipulated growth. Manipulation can be achieved by methods well known in the art, for example, by removing or failing to produce one or more nutrients necessary for cell multiplication.

Although the PHA-producing organism need not be a microbial organism, in certain embodiments such microbial organisms are preferred. Numerous microbial organisms are known in the art to be suitable for the production of PHA polymers (see, e.g., Anderson and Dawes, Micro. Rev. 54 (4): 450 472, 1990). The microbial organisms may be wild type or mutated or may have the necessary genetic material introduced into it, for example by methods well known in the art, e.g., recombinant DNA technology (see, e.g., Nomura, C. T., and S. Taguchi. 2007. PHA synthase engineering toward superbiocatalysts for custom-made biopolymers. Appl Microbiol Biotechnol 73:969-79).

Genes suitable for introduction and well known in the art include those described in Ahn, I. S., W. C. Ghiorse, L. W. Lion, and M. L. Shuler. 1998. Growth kinetics of Pseudomonas putida G7 on naphthalene and occurrence of naphthalene toxicity during nutrient deprivation. Biotechnology and Bioengineering 59:587-594; Nomura, C. T., and S. Taguchi. 2007. PHA synthase engineering toward superbiocatalysts for custom-made biopolymers. Appl Microbiol Biotechnol 73:969-79; Steinbuchel, A., and S. Hein. 2001. Biochemical and molecular basis of microbial synthesis of polyhydroxyalkanoates in microorganisms. Adv Biochem Eng Biotechnol 71:81-123.

Determination of new PHA monomer structures can be accomplished using methods well known in the art, such as Nuclear Magnetic Resonance (NMR) Spectroscopy (see, e.g., Nomura, C. T., K. Taguchi, S. Taguchi, and Y. Doi. 2004. Coexpression of genetically engineered 3-ketoacyl-ACP synthase III (fabH) and polyhydroxyalkanoate synthase (phaC) genes leads to short-chain-length-medium-chain-length polyhydroxyalkanoate copolymer production from glucose in Escherichia coli JM109. Appl Environ Microbiol 70:999-1007; Nomura, C. T., et al. 2008. FabG mediates polyhydroxyalkanoate (PHA) production from both related and nonrelated carbon sources in recombinant Escherichia coli LS5218. Biotechnol Prog. In press, American Chemical Society and American Institute of Chemical Engineers, published on web Jan. 24, 2008; Nomura, C. T. et al. 2004. Effective enhancement of short-chain-length (SCL)-medium-chain-length (MCL) polyhydroxyalkanoate copolymer production by co-expression of genetically engineered 3-ketoacyl-acyl-carrier protein synthase III (fabH) and polyhydroxyalkanoate synthesis genes. Biomacromolecules 5:1457-1464).

PHAs comprise hydroxyalkanoate (HA) monomers, which are substrates for PHA synthase enzymes. Biologically-produced PHA polymers are the product of PHA synthase microbial enzymes, and are produced using methods well known in the art, in either a bacterial cell that naturally comprises a PHA synthase, or in a bacterial or other cell type, for example a plant cell, that has been genetically engineered to express such an enzyme. Microbial PHA synthase enzymes are known in the art to have broad substrate ranges and to be capable of incorporating a large number of HA monomers as constituents of biosynthetic PHA depending upon growth conditions, precursor substrate availability, and the source of the PHA synthase enzyme.

Suitable HA monomers for producing PHAs are well known in the art, see, e.g., U.S. Pat. No. 7,208,535 (PHA compositions and methods for their use in the production of PHA films, Asrar et al., issued Apr. 24, 2007).

Many genera of microbes are known in the art that can be used for producing PHAs (see, e.g., U.S. Pat. No. 5,516,688) including but not limited to: Escherichia (e.g., E. coli), Pseudomonas, Alcaligenes; Corynebacterium, Acinetobacter, Rhodococcus, Nocardia, Mycobacterium, Bacillus, Flavobacterium, Cunnighamella, Agmenellum, Phanerochaete, Burkholderia, Sphingomonas, Vibrio, Comamonas, Ralstonia, and Gordona. In addition, PHAs can be produced in eukaryotic systems such as yeast (Saccharomyces, Pichia, etc.) or plants (Arabidopsis, switch grass, sugar cane, etc.).

The absorption rate and efficiency of PHA monomers and/or polymers can be quantified using methods well known in the art. New or known PHA monomer or polymer structures can be tested, using methods well known in the art, for their ability to bind, capture or uptake HOCs. For example, standard methods of gas chromatography and mass spectrometry (GC/MS) can determine polymer absorption efficiency. A standard curve can be constructed based on the GC/MS results from a standard of known HOC concentration, e.g., HOC dissolved in hexanes.

In one embodiment, the efficiency of hexanes in extraction of a HOC such as naphthalene from aqueous solution can be ascertained based on a standard curve. Biodegradable polymers can then be added to an aqueous solution of known naphthalene concentration, followed by a hekanes extraction of the polymers. The remaining aqueous solution can be extracted with hexanes for comparison with the polymer extricant. Extraction rates are measurable through extractions conducted over increasing periods of time.

Other standard methods well known in the art for quantifying absorption rate and efficiency of PHA monomers and/or polymers are disclosed, for example, in Huckins, J. N., J. D. Petty, J. A. Lebo, C. E. Orazio, and D. E. Prest (1997. Accumulation of organochlorine pesticides and PCBs by semipermeable membrane devices and Mytilus edulis in New Bedford Harbor—Comment. Environmental Science & Technology 31:3732-3733) and in van Stee, L. L. P., P. E. G. Leonards, W. M. G. M. van Loon, A. J. Hendriks, J. L. Maas, J. Struijs, and U. A. T. Brinkman (2002. Use of semi-permeable membrane devices and solid-phase extraction for the wide-range screening of microcontaminants in surface water by GC-AED/MS. Water Research 36:4455-4470).

### 5.4 Organisms that Degrade HOCs and that Can Be Used as Bioremediation Vectors

Many genera of microbes are known in the art that can be used for degrading HOCs (see, e.g., U.S. Pat. No. 5,516,688) including but not limited to: Escherichia (e.g., E. coli), Pseudomonas, Alcaligenes; Corynebacterium, Acinetobacter, Rhodococcus, Nocardia, Mycobacterium, Bacillus, Flavobacterium, Cunnighamella, Agmenellum, Phanerochaete, Burkholderia, Sphingomonas, Vibrio, Comamonas, and Gordona.

Bacteria that can degrade 2-3 ring low molecular weight PAHs, such as naphthalene, phenanthrene, bi-phenyl and fluorene, are well known in the art, and include, but are not limited to gram-negative genera listed above such as Pseudomonas, Burkholderia, Alcaligenes, Sphingomonas, Vibrio and Comamonas.

Gram-positive genera such as Mycobacterium, Nocardia, Rhodococcus and Gordona listed above are also known to degrade low molecular weight PAHs.

Microorganisms that can degrade the 4-ring and higher high molecular weight PAHs such as pyrene, fluoranthene and benz[a]anthracene are also known in the art, and include, but are not limited to Paenibacillus species such as P. validus (U.S. Pat. No. 6,503,746, Daane et al., issued Jan. 7, 2003).

Conditions for growing or maintaining bacterial strains on substrates or in biomasses for use in bioremediation are well known in the art (see, e.g., U.S. Pat. No. 6,503,746).

### 5.5 Methods for Determining the Capability of Microbial Organisms to Metabolize Absorbed and Unabsorbed PAHs

The efficiency at which a selected microbe utilizes a polymer/PAH complex for metabolism, regenerating the original biodegradable polymer, can be observed using methods well known in the art (see, e.g., Ahn, I. S., W. C. Ghiorse, L. W. Lion, and M. L. Shuler. 1998. Growth kinetics of Pseudomonas putida G7 on naphthalene and occurrence of naphthalene toxicity during nutrient deprivation. Biotechnology and Bioengineering 59:587-594; Velazquez, F., V. de Lorenzo, and M. Valls. 2006. The m-xylene biodegradation capacity of Pseudomonas putida mt-2 is submitted to adaptation to abiotic stresses: evidence from expression profiling of xyl genes. Environ Microbiol 8:591-602).

### 5.6 Applications and Apparatuses Utilizing PHA Polymers

Numerous applications and apparatuses can use PHA polymers. PHA polymers can be used, as described herein, for bioremediation, e.g., the removal of HOC contaminants or pollutants from gases, gaseous fluids, fluids or solid materials. For example, PHA polymers can be used in filters (see, e.g., U.S. Pat. No. 6,792,953, Tobacco smoke filter Lesser et al. Sep. 21, 2004).

The methods and apparatuses provided herein can also be used in medical devices and applications, e.g., See, e.g., U.S. Pat. No. 7,268,205, Medical devices and applications of polyhydroxyalkanoate polymers, Williams et al., Sep. 11, 2007.

PHA polymers can be used in devices and applications for solid phase microextraction and desorption (see, e.g., U.S. Pat. No. 6,042,787, Device for solid phase microextraction and desorption, Pawliszyn, Mar. 28, 2000). For example, PHA polymers can be used as an aqueous filtration device for the removal of HOCs. They can also be used as a gaseous filtration device to remove HOCs.

PHA polymers can be used to remove HOCs from solids, e.g., soil or organic matter. The solid containing (or contaminated with) an HOC can be contacted with a PHA that has affinity for the HOC. For example, the PHA can be plowed into soil, or stirred into or added to a mixture or slurry of solids suspended in a liquid, whereby it binds or sequesters the HOC.

In one embodiment, an HOC can be in equilibrium in an aqueous or liquid environment with a contaminated solid material and a PHA that has higher affinity for the HOC than the solid material. Methods of determining affinity of a PHA for an HOC are well known in the art. The PHA, since it has higher affinity, will absorb or remove more HOC from the solid material than the solid material itself will.

In another embodiment, an apparatus is provided for adsorbing a HOC from a material contaminated with the HOC comprising a PHA polymer that binds with at least one HOC, wherein the PHA polymer is disposed in the apparatus in an orientation so that it contacts the material contaminated with the HOC.

In another embodiment, the PHA polymer (e.g., a PHA in the form of a fiber or film) is loaded on a support.

The apparatus can maintain environmental conditions to sustain HOC adsorption or sequestration until a desired amount of adsorption or sequestration of the HOC is achieved. The apparatus preferably does not disturb current or existing environmental conditions at the site at which (or in the material in which) testing, detection or remediation is being conducted. The apparatus preferably allows environmental conditions such as ambient water flow, moisture level, temperature, and pH to be maintained and remain undisturbed at the site (or in the material) tested. For example, the apparatus preferably does not disturb existing pH conditions or does not release other substances (e.g., environmental pollutants or toxins) into the environment being tested, and removes only the targeted molecule or contaminant (e.g., a HOC or PAH) from the environment (or material) being tested.

In specific embodiments, the apparatus can be a filter, wand, cone or tube.

In another embodiment, the apparatus is a coating.

In another embodiment, the apparatus support is selected from the group consisting of metal rod, metal tube, metal mesh, glass rod, glass tube or glass mesh.

In another embodiment, the support is a gelatinous medium.

In another embodiment, the support is selected from the group consisting of gels, pastes, paper, and aqueous solutions. In another embodiment, the support is a filter.

An apparatus is also provided for detecting a HOC in a material comprising:

a PHA polymer that binds with at least one HOC, wherein:
the PHA polymer is disposed in the apparatus in an orientation so that it contacts the material;
the PHA polymer binds with the HOC in the material; and
the presence of the HOC in the PHA polymer is determined by performing an assay for the HOC.

In one embodiment, the assay comprises determining a concentration of the HOC bound with the PHA polymer.

In one embodiment, the assay comprises performing gas chromatography and mass spectroscopy (GC/MS).

The following examples are offered by way of illustration and not by way of limitation.

### 6. Examples

### 6.1 Example 1

### Adsorption of PAH Naphthalene by PHA

6.1.1 Introduction

This example describes an experiment that was performed to assay the ability of a PHA to adsorb the PAH naphthalene at various concentrations from an aqueous solution. Naphthalene was detected by gas chromatography/mass spectrometer (GC/MS). The results show that the use of PHA is advantageous, owing to its ability to concentrate hydrophobic compounds. The use of PHA can therefore provide a platform for microbial remediation of these compounds. In addition, as these data show, PHA polymers can be used to easily detect microcontaminant levels as low as 0.01 ppm of HOCs without using large solvent volumes.

6.1.2 Materials and Methods

Polymer Production

PHA polymers were produced by a recombinant E. coli strain harboring a plasmid expressing the phaC1STQK gene with fabH(F87T), and phaAB and the polymer content and composition was checked by gas chromatography. These methods are known in the art and have been previously described by Nomura, C. T., K. Taguchi, S. Taguchi, and Y. Doi (2004. Coexpression of genetically engineered 3-ketoacyl-ACP synthase III (fabH) and polyhydroxyalkanoate synthase (phaC) genes leads to short-chain-length-medium-chain-length polyhydroxyalkanoate copolymer production from glucose in Escherichia coli JM109. Appl Environ Microbiol 70:999-1007). Methods for synthesizing S-containing PHA compound are described in Lutke-Eversloh, T., K. Bergander, H. Luftmann, and A. Steinbuchel (2001. Biosynthesis of poly(3-hydroxybutyrate-co-3-mercaptobutyrate) as a sulfur analogue to poly(3-hydroxybutyrate) (PHB). Biomacromolecules 2:1061-5).

Polymer Purification

The polymer was extracted from 30-40 g of lyophilized cells in 2000 ml of chloroform over 24 h. Cells debris and other insoluble material was removed by filtration through Whatman filter paper No. 1 followed by filtration through a PTFE membrane. The chloroform was evaporated from the filtered polymer solution via rotary evaporation and the polymer was washed with methanol and removed and dried at room temperature in a fume hood for 1 h. The polymer was redissolved in 400 ml of chloroform, filtered again through a PTFE membrane and heated to 100° C. in a pressure-resistant glass vial in an oil bath for 5-12 hours. PHA fibers were made by slowly dripping (˜0.1 ml per min) the polymer solution into 4 l of hexane with stirring. The fibers were collected by filtration with Whatman filter paper No. 1. The fibers were washed with methanol and allowed to dry at room temperature.

Hexanes Standard Preparation

A 20 ppm naphthalene hexanes solution was made by adding hexanes (Fisher Scientific, USA) to naphthalene (98% Aldrich, USA). The stock 20 ppm naphthalene hexanes solution was serial diluted to concentrations of 0.01 ppm, 0.1 ppm, 1 ppm, 2 ppm, 5 ppm and 10 ppm in 2 ml amber vials (National Scientific, USA). A 2 ppm internal anthracene (99.9% Aldrich, USA) solution was added to the vials before analyzed on Gas Chromatography/Mass Spectrometer (GC/MS).

Sample Preparation for Pre-Polymer Treatment

A 20 ppm naphthalene aqueous solution was made by adding miliQ ddH2O to naphthalene (98% Aldrich, USA). The naphthalene miliQ ddH2O mixture was sonicated over approximately eight hours at 55° C. until all naphthalene went into solution. The stock naphthalene aqueous solution was serial diluted to concentrations of 0.01 ppm, 0.1 ppm, 1 ppm, 2 ppm, 5 ppm and 10 ppm in 40 ml amber vials (VWR TraceClean Quality-Assured vials, USA). Hexanes was added to each vials and allowed to incubate with shaking overnight at room temperature. The shaker (Taitec Recipro Shaker, Japan) was set at 200 rpm. The hexanes extractants were taken out via glass pipettes into 2 ml amber vials. An internal standard of 2 ppm anthracene was added to the hexanes prior analysis on GC/MS.

Polymer Treatment Sample Preparation

Polymer films were obtained from dissolving the polymer fibers in chloroform and allowing the chloroform to evaporate over approximately 24 hours at room temperature. A set of naphthalene aqueous solution with concentrations of 0.01 ppm, 0.1 ppm, 1 ppm, 2 ppm, 5 ppm, and 10 ppm was made in 40 ml amber vials by serial dilution from the 20 ppm aqueous naphthalene stock solution. Approximately 30 mg polymer fibers or films were added directly to the naphthalene solutions. First, the polymer and aqueous naphthalene mixtures were incubated with shaking over night at 200 rpm room temperature. Then, the naphthalene aqueous solutions were transferred via glass pipettes to another set of 40 ml amber vials. Both naphthalene aqueous solutions and the polymers were extracted with hexanes over night by shaking at room temperature on a shaker at 200 rpm. The hexanes extractants were transferred via glass pipettes into 2 ml amber vials. An internal standard of 2 ppm anthracene was added to the extractants prior GC/MS analysis.

Gas Chromatography/Mass Spectrometer Analysis

The amounts of naphthalene before and after treatment with polymer were determined by GC/MS on a Hewlett Packard model 5989B (USA) (at A&TS, Analytical & Technical Services, State University of New York, College of Environmental Science and Forestry, Syracuse N.Y.). A volume of 30 μl of each extractants was auto-injected into the GC/MS. The naphthalene and anthracene peaks were identified by their mass/charge ratio. The amounts of naphthalene presented in the extractants were determined by using their absolute abundance measured. The injection syringe was rinsed three times each with hexanes, chloroform and acetone prior and after sample injection.

6.1.3 Results

PHA Fibers and Films

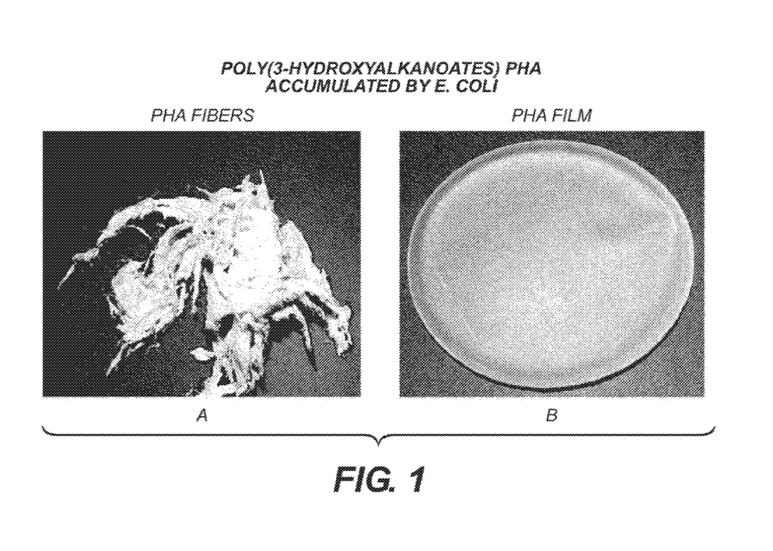

PHA polymer composed of poly(3-hydroxybutyrate) purified from recombinant E. coli as determined by GC and NMR analysis was used for the experiments. Polymers used for the experiment are shown in FIG. 1, although other types of PHA polymers composed of poly-3-alkanoates (PHA or P(3HA)) such as 3-hydroxypropionate, 3-hydroxyhexanoate, 3-hydroxyoctanoate, 3-hydroxydecanoate, 3-hydroxynonoate, 3-hydroxydodecanoate, 3-hydroxytetradecanoate, 3-hydroxyvalerate, 4-hydroxyalkanoates such as 4-hydroxybutyrate and 4-hydroxyvalerate, 5-hydroxyvalerate, 6-hydroxyhexanoate, phenoxyalkanoates, or other derivatives are expected to have different affinities toward PAHs and other aromatic compounds such as PCBs, dependent on their structure. These polymers can be of a wide range of molecular weights from 20,000 to 2×10⁶. For this experiment the polymers had molecular weights of 5×10⁵. Once the film or fibers were purified, they were used as described in the Materials and methods.

Gas Chromatography/Mass Spectrometer Analysis

Results of the experiment have shown that the PHA polymer can be used to detect 0.01 ppm (nM range) of naphthalene and the ability to adsorb up to 80% of the naphthalene in the solution upon a single treatment with polymer. This is a level of detection comparable to current techniques in the field.

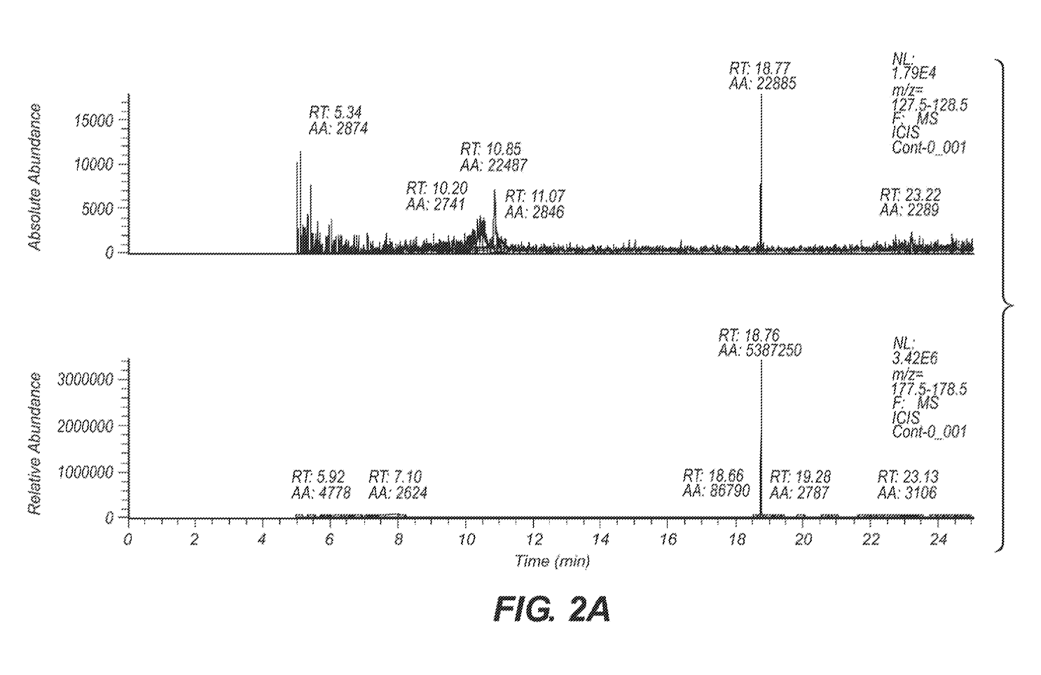

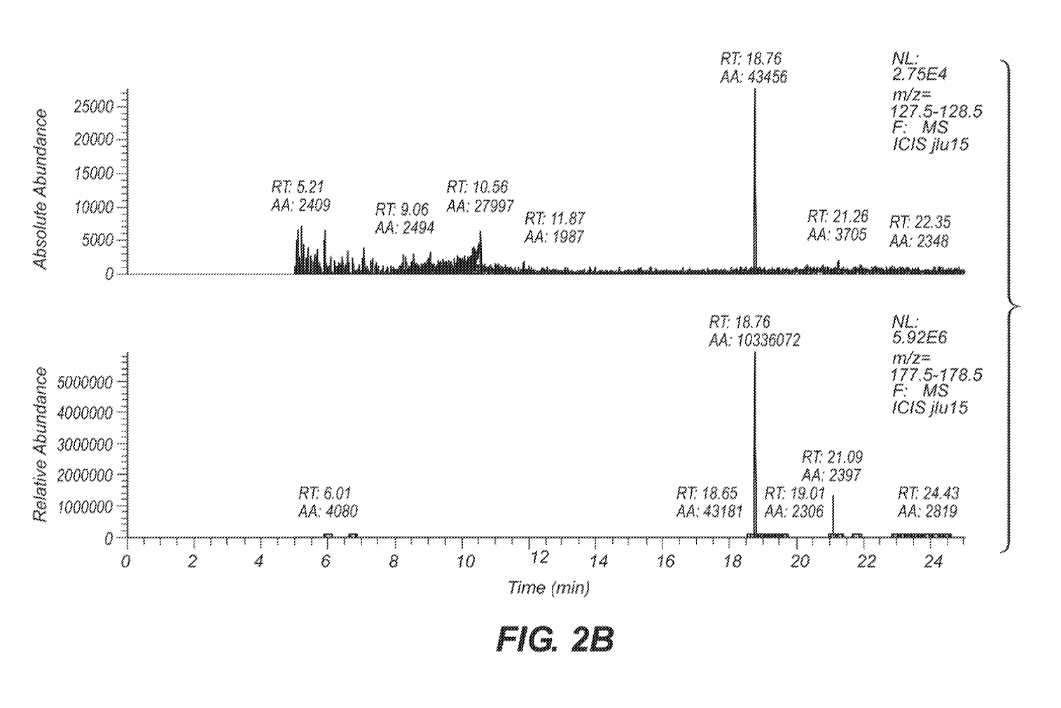

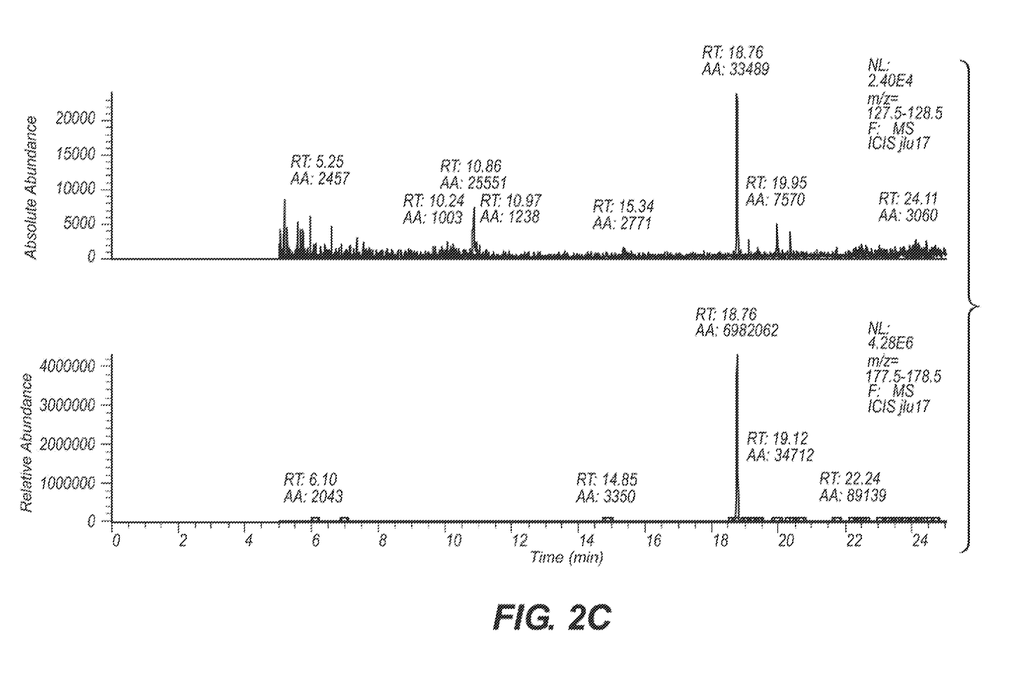

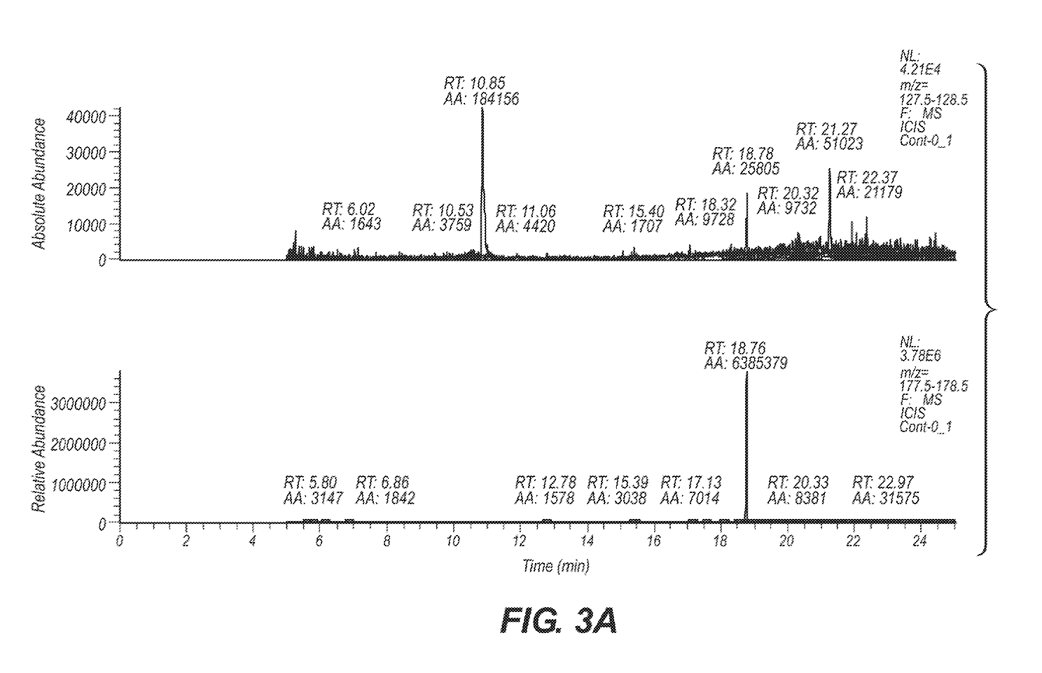

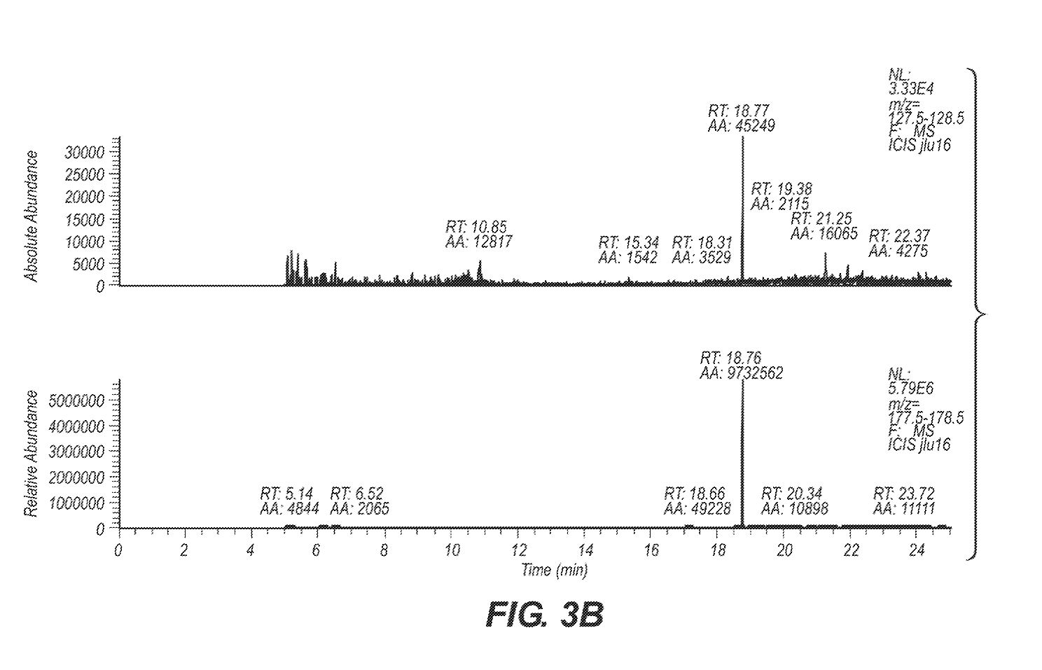

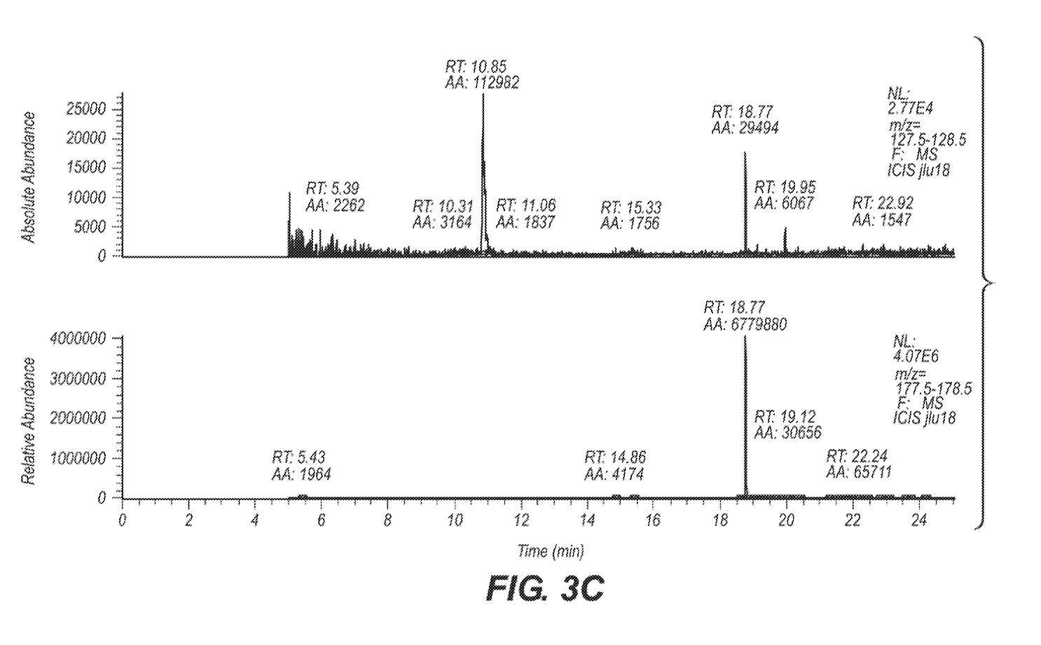

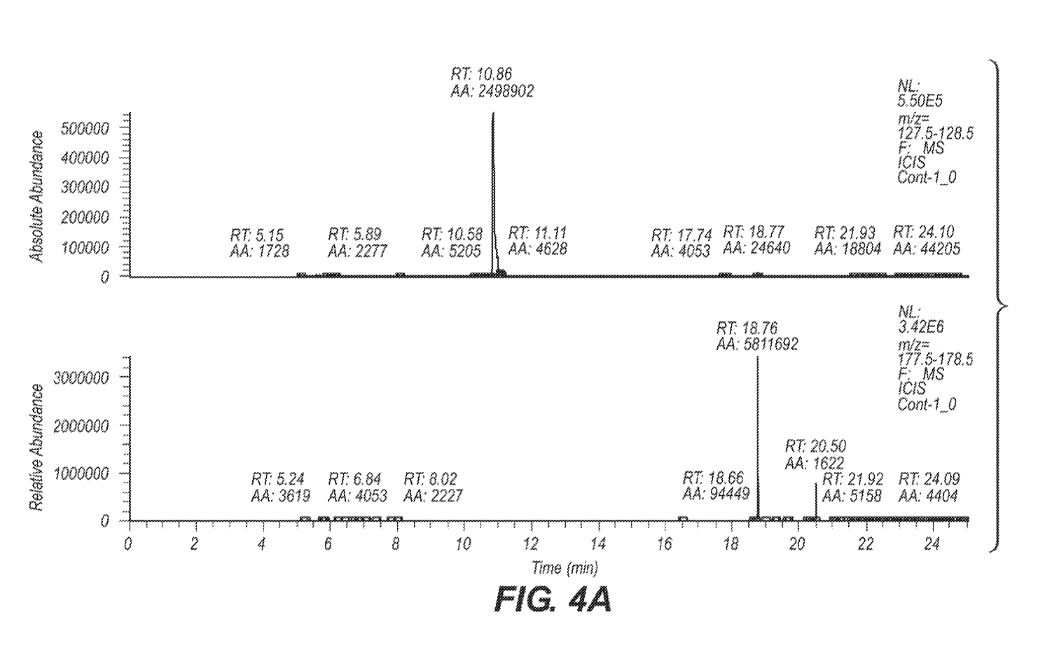

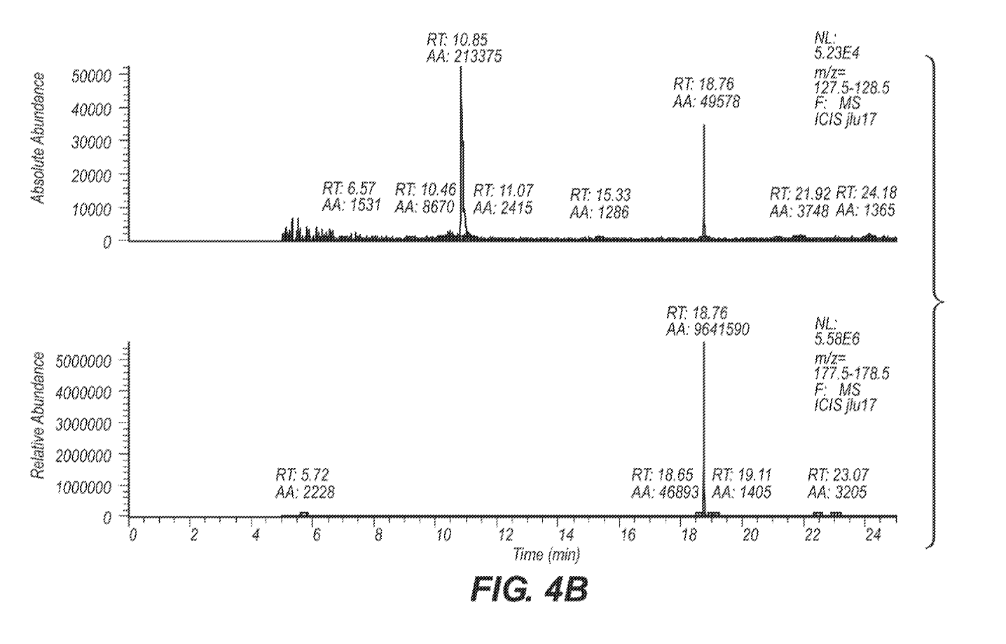

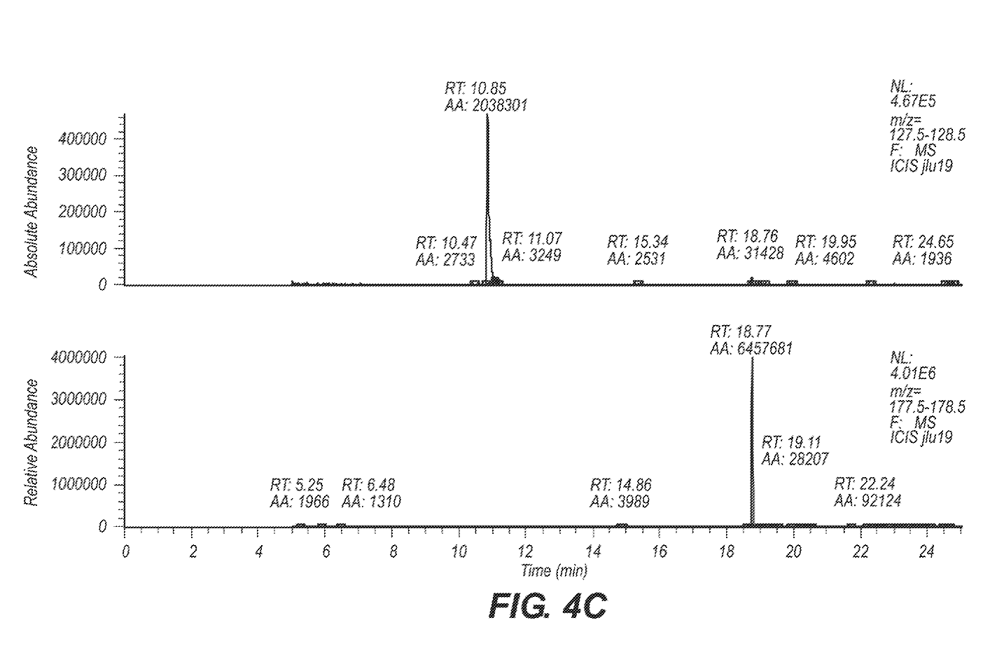

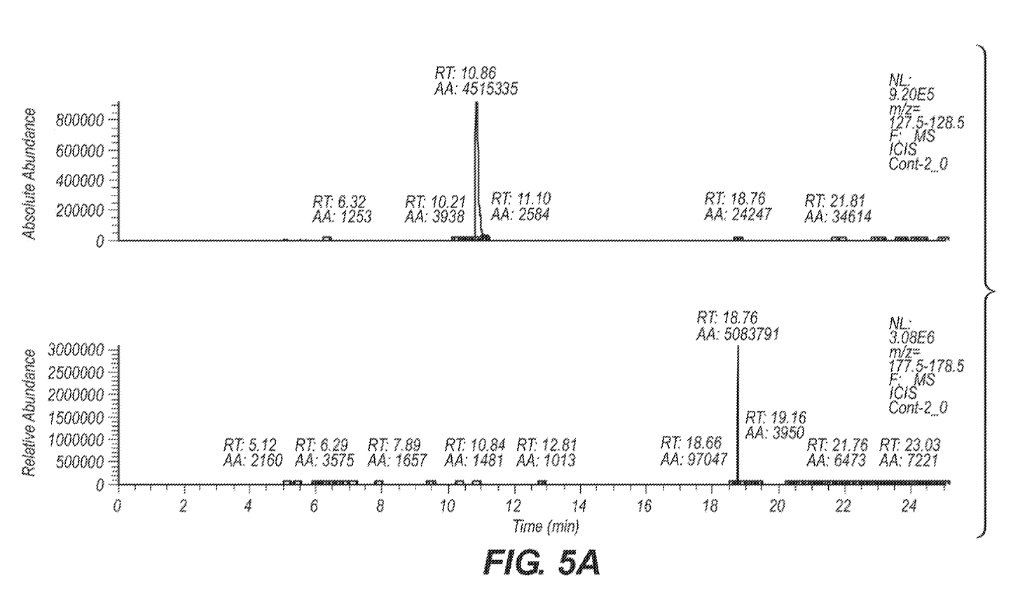

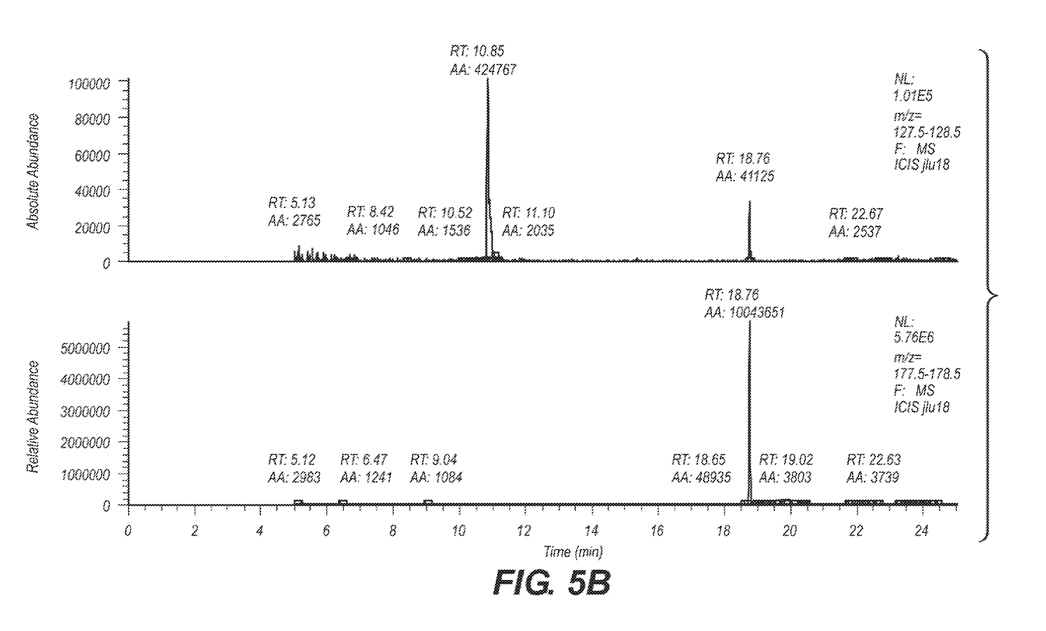

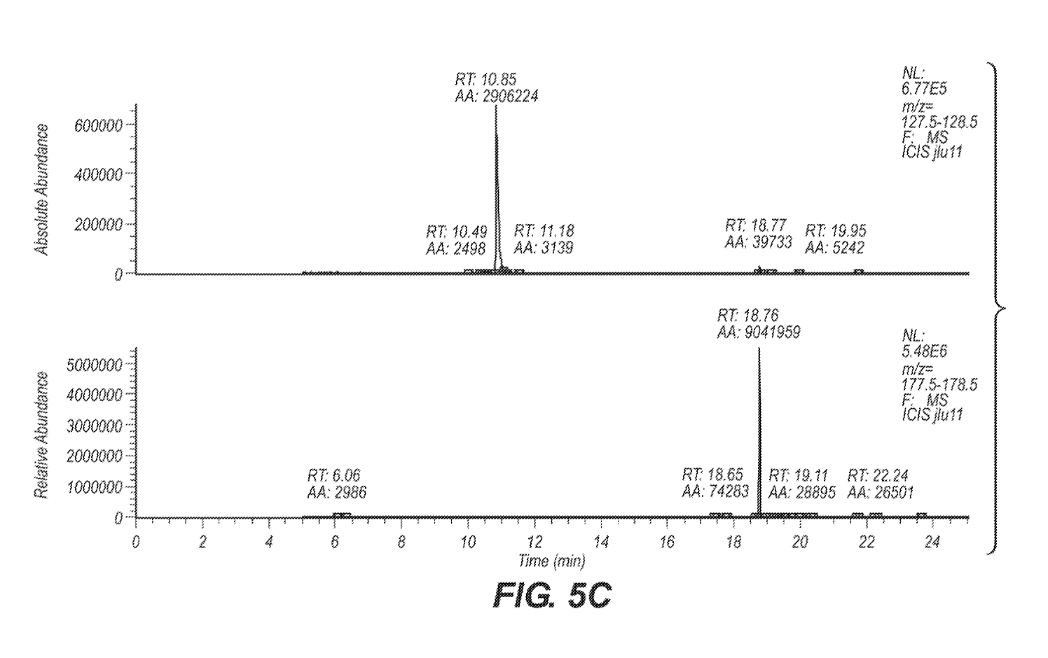

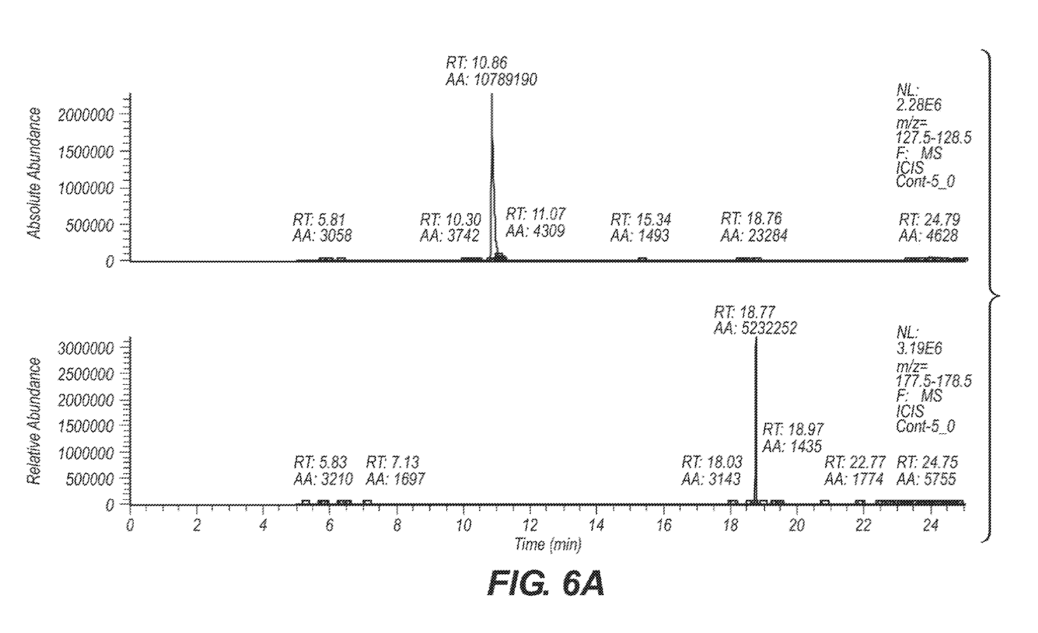

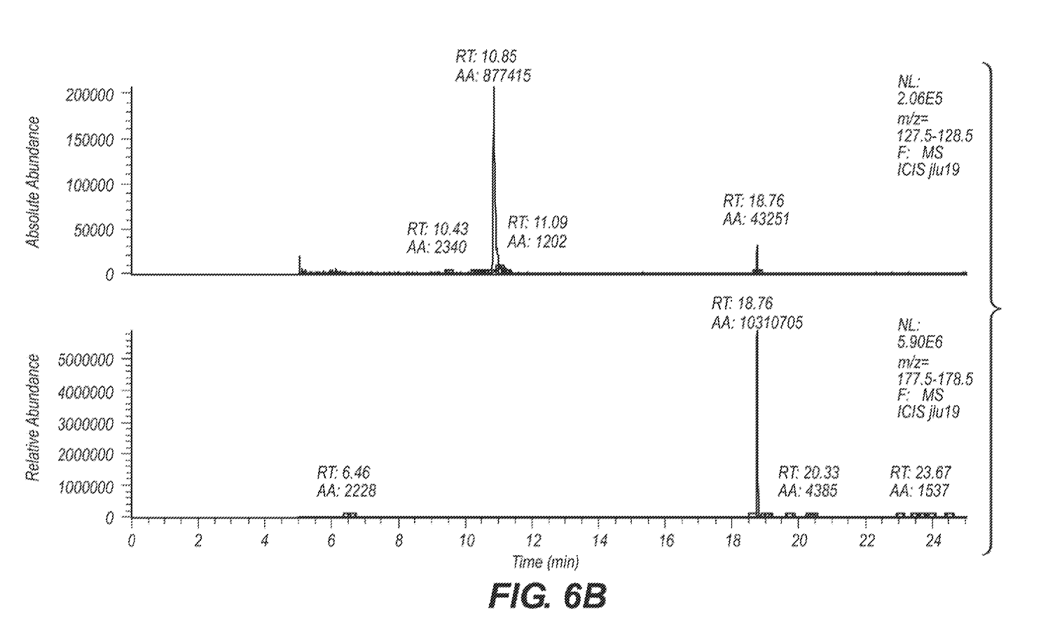

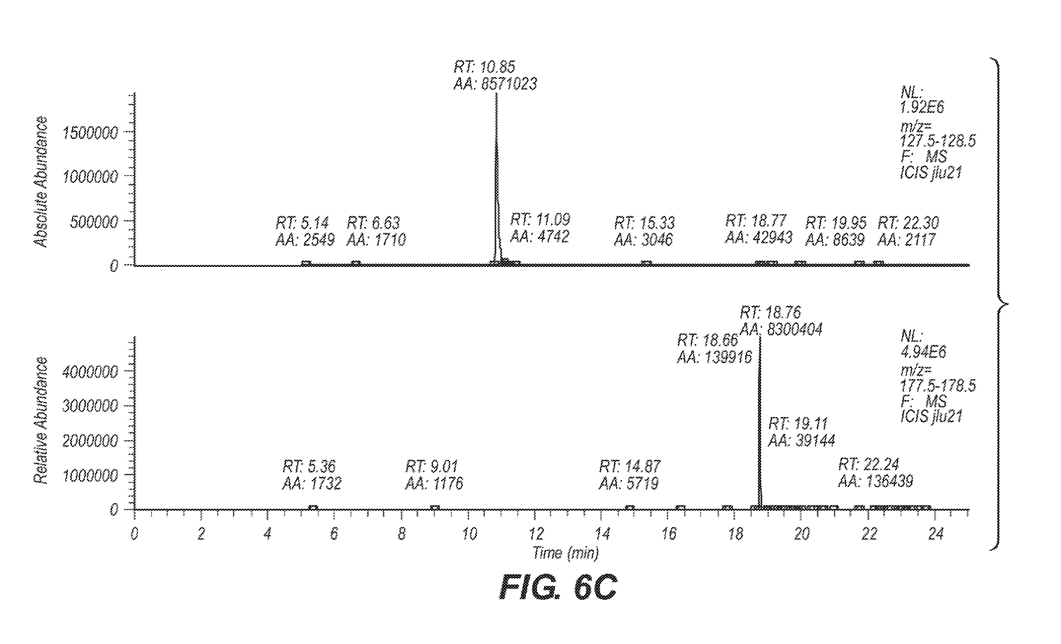

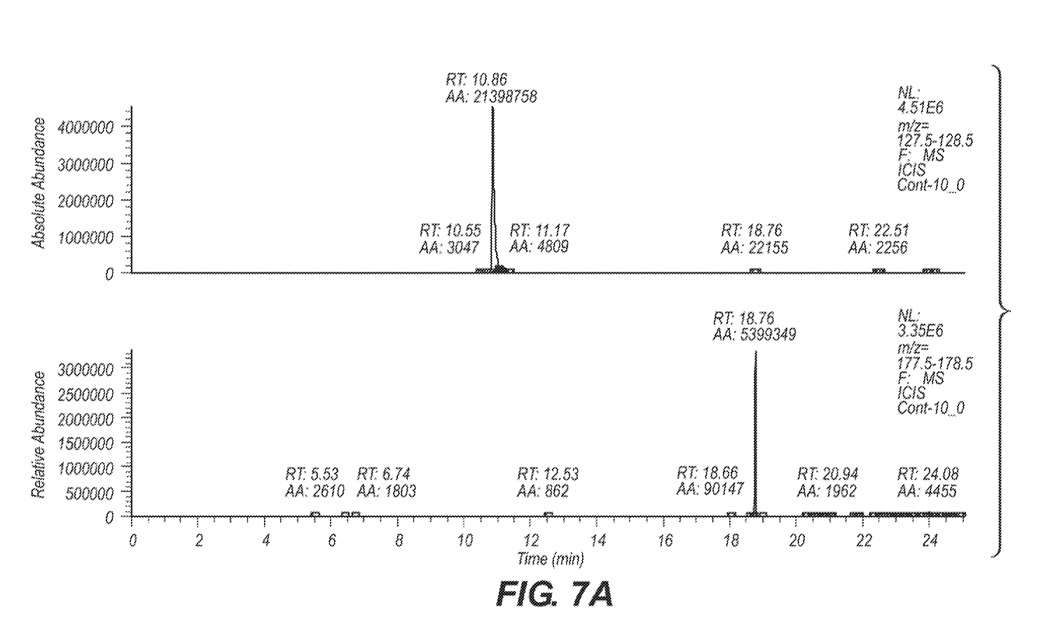

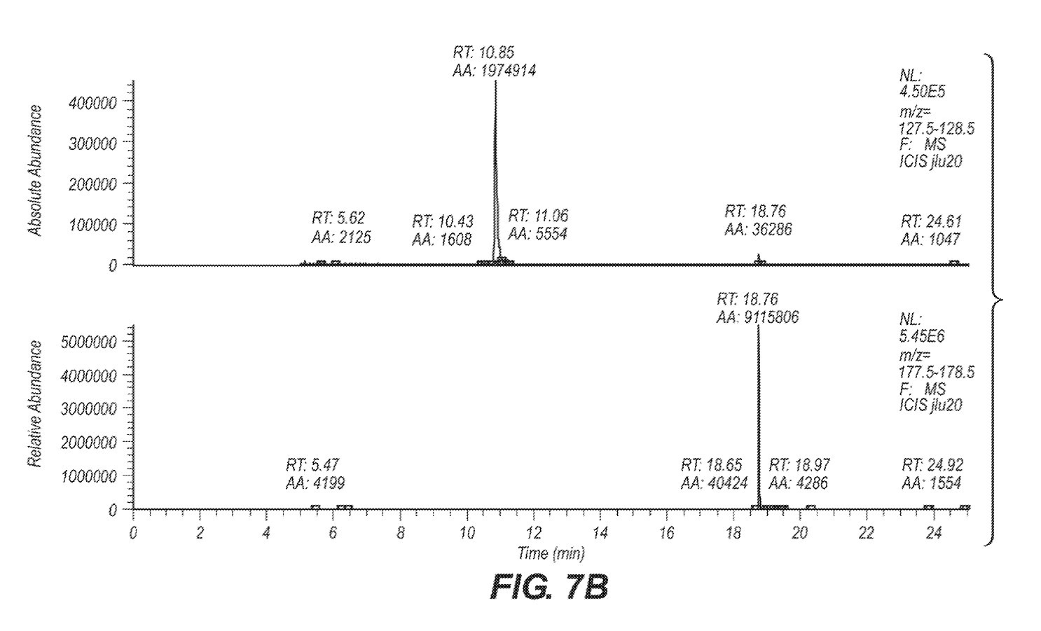

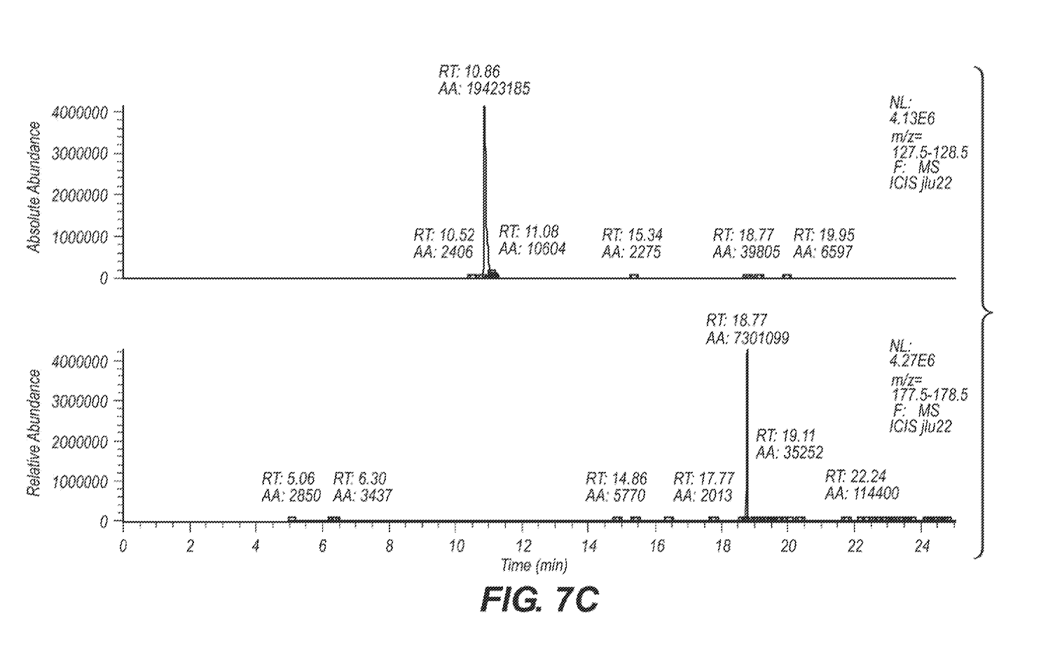

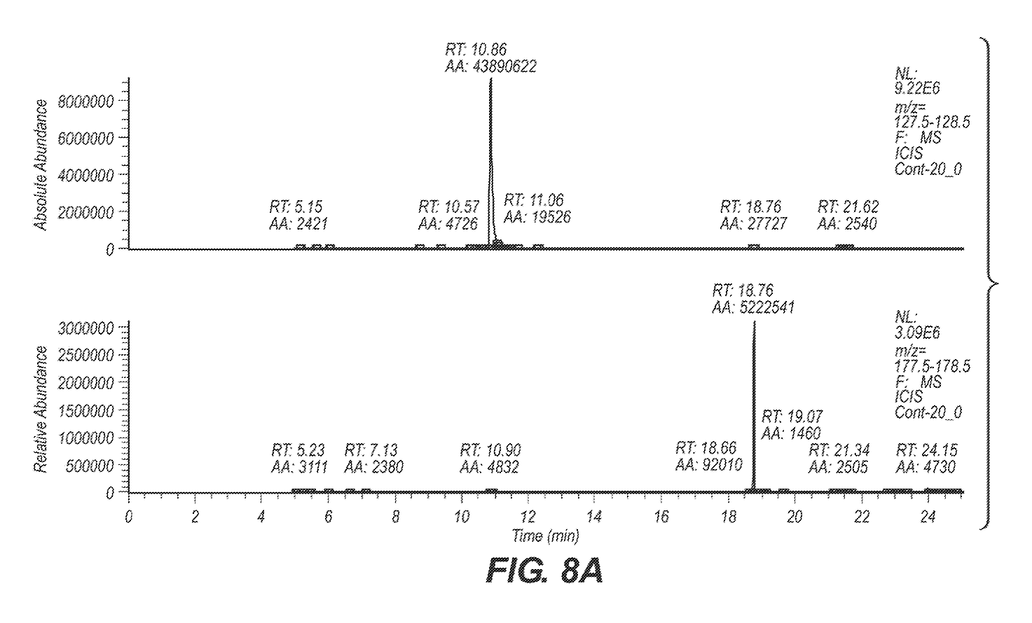

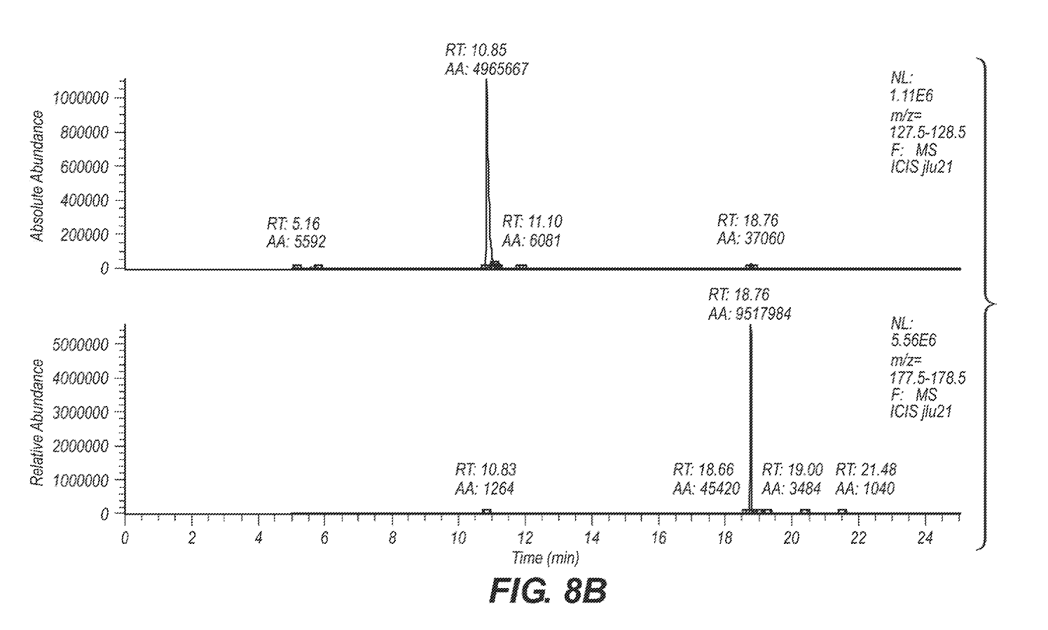

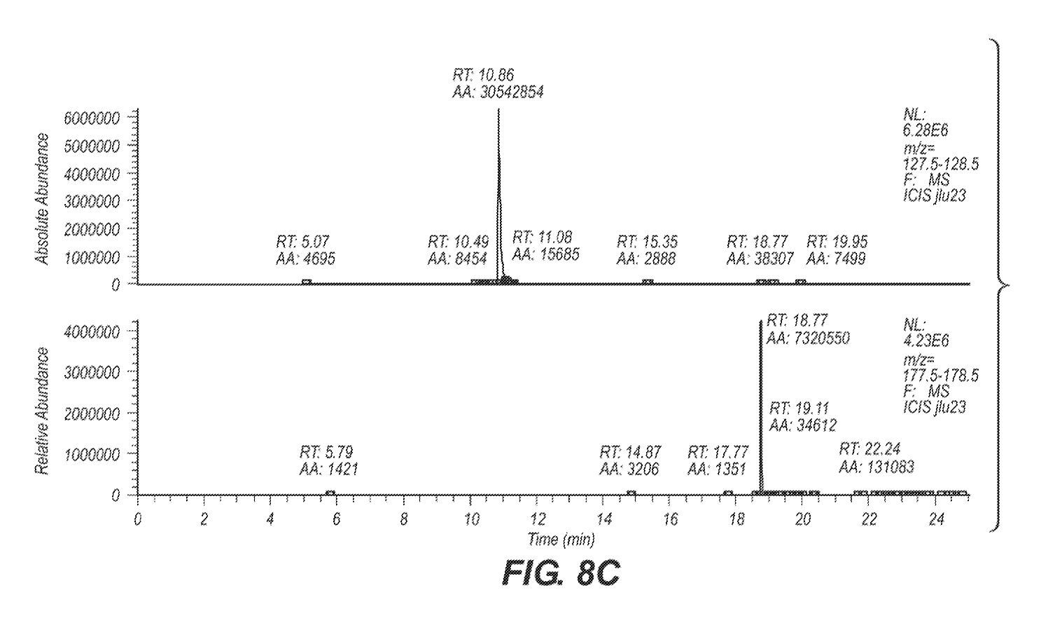

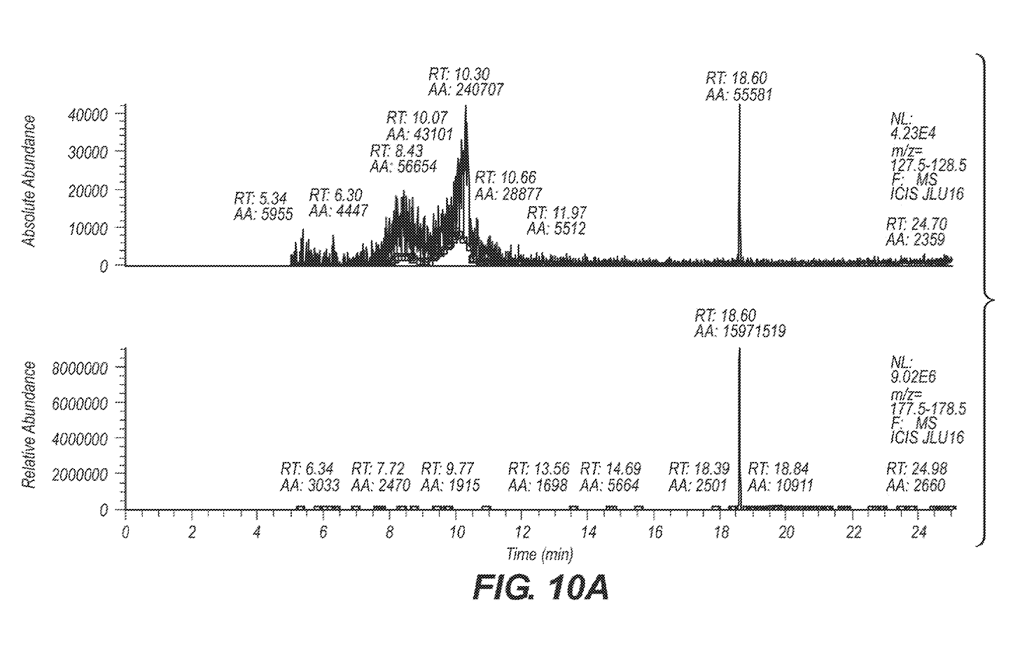

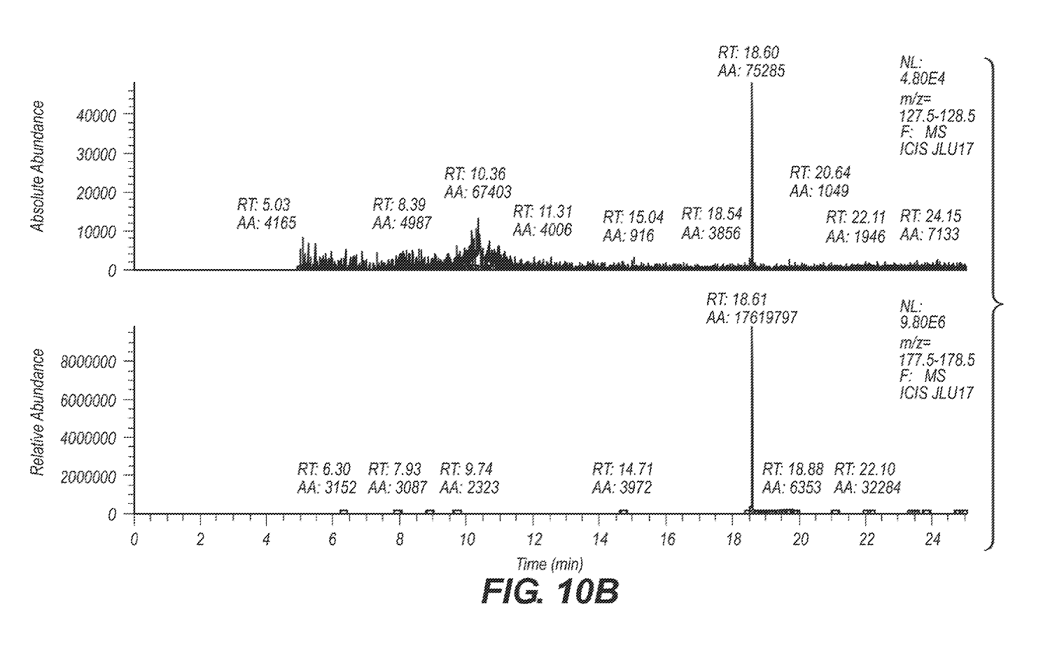

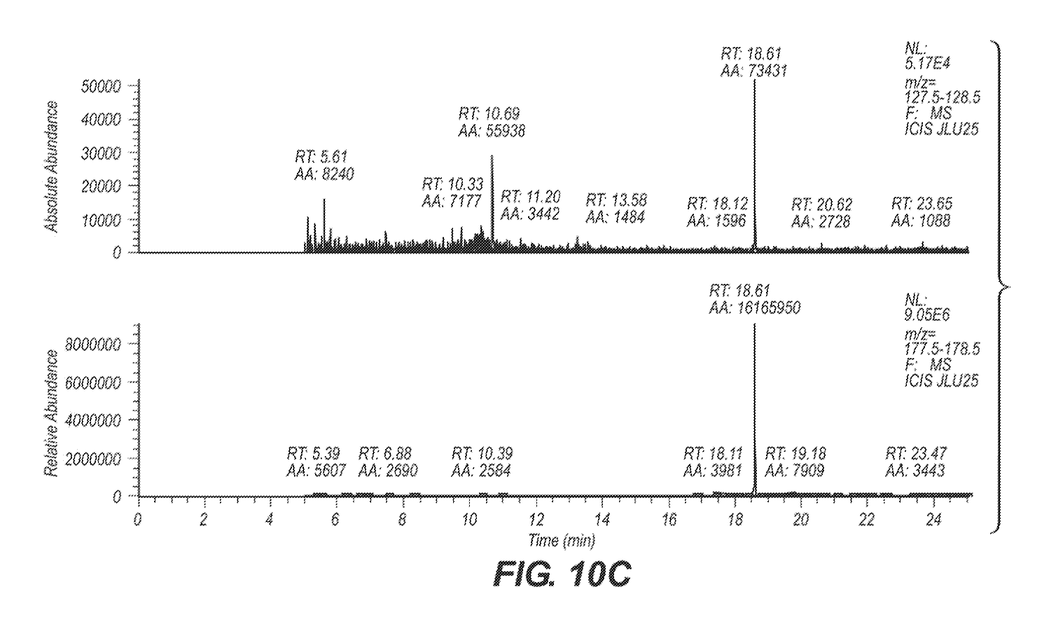

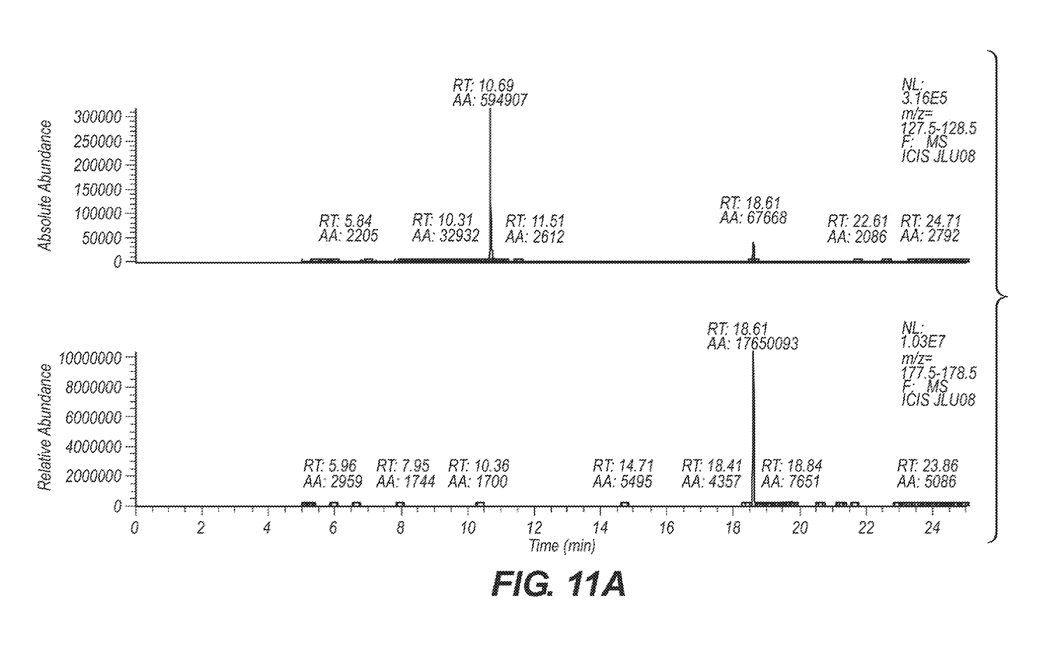

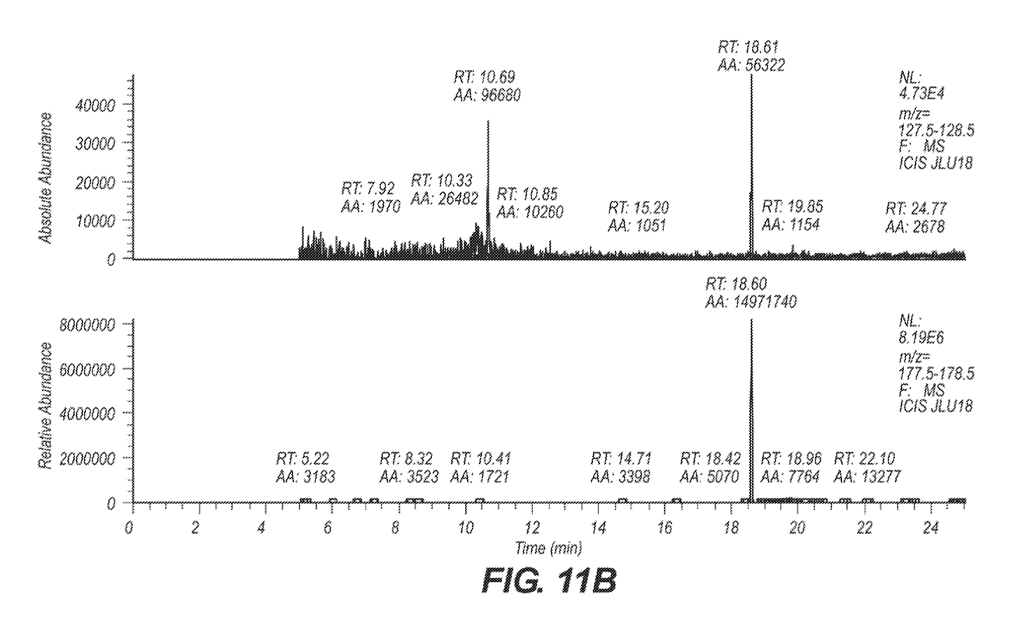

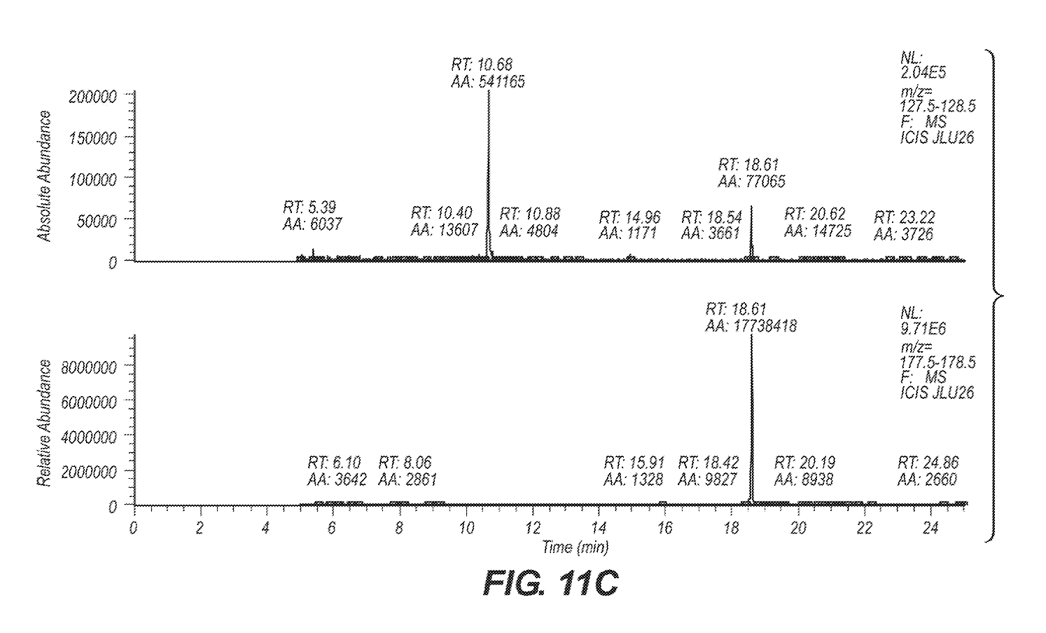

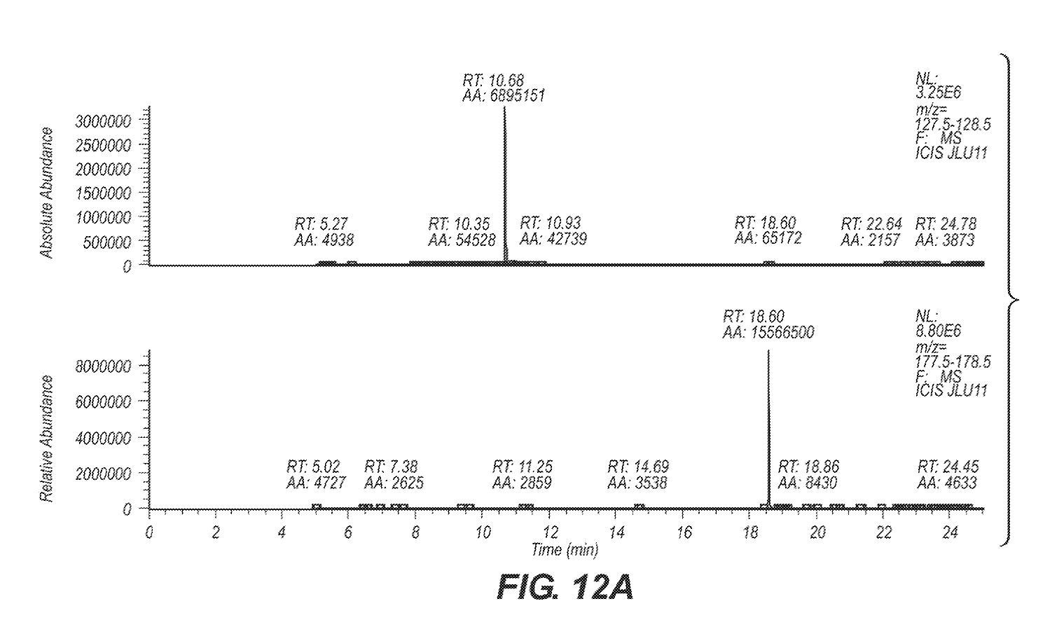

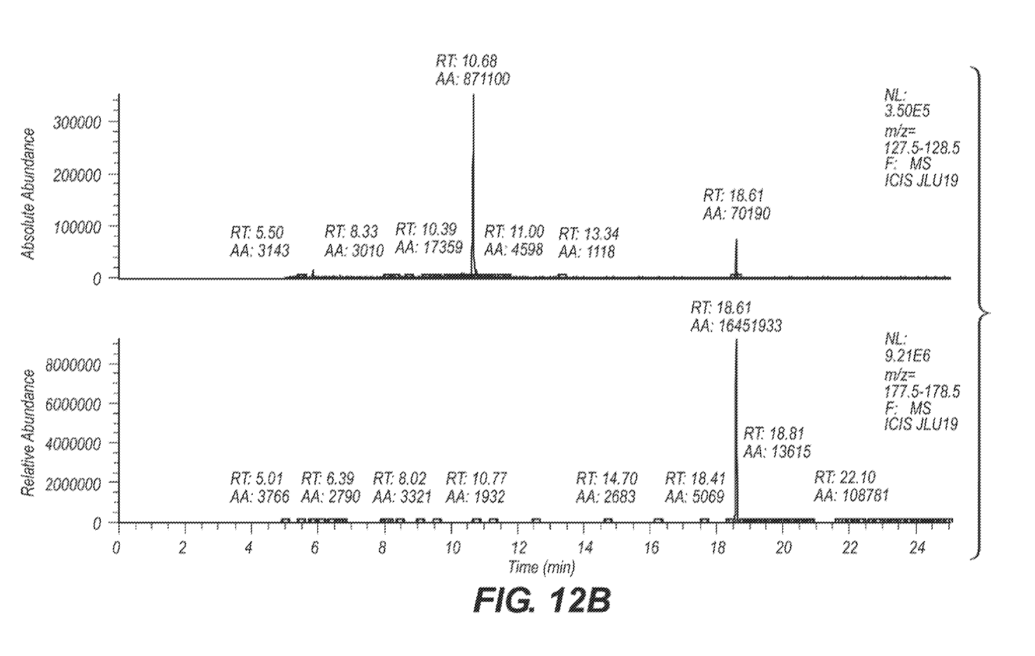

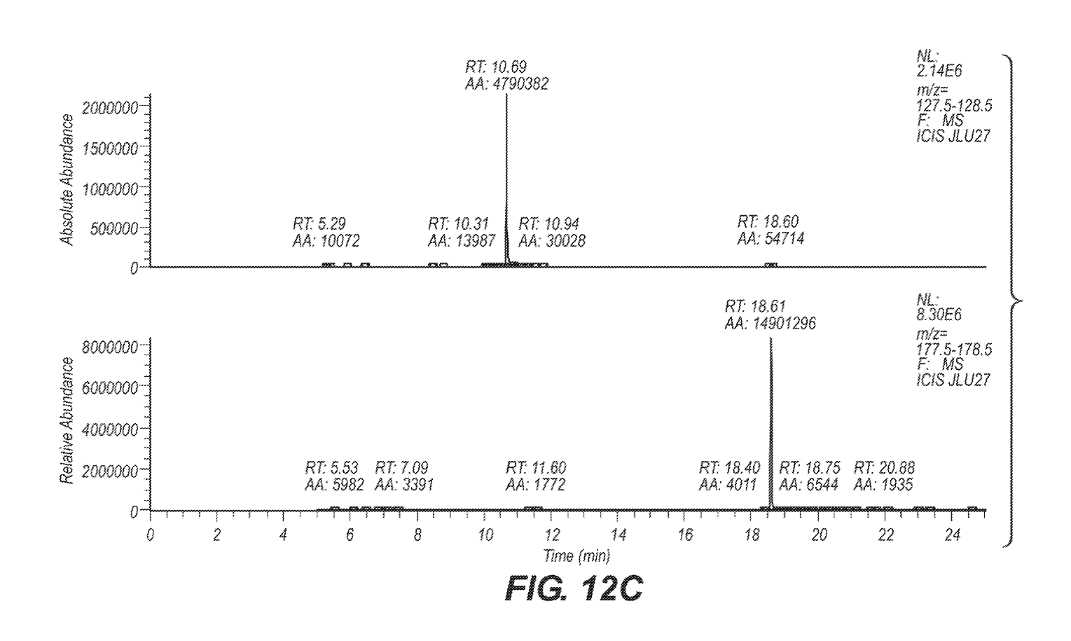

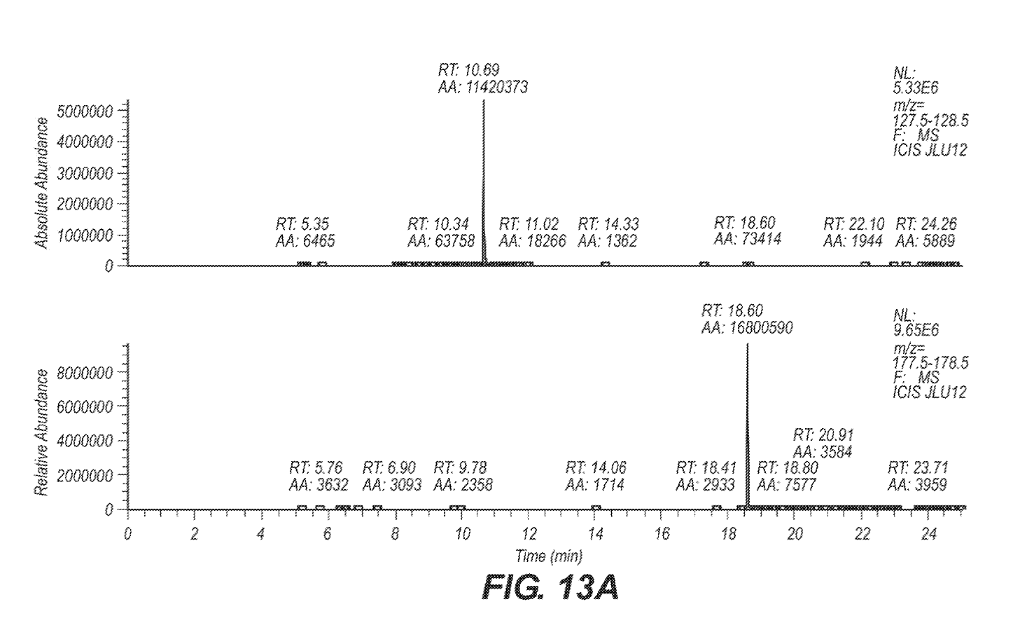

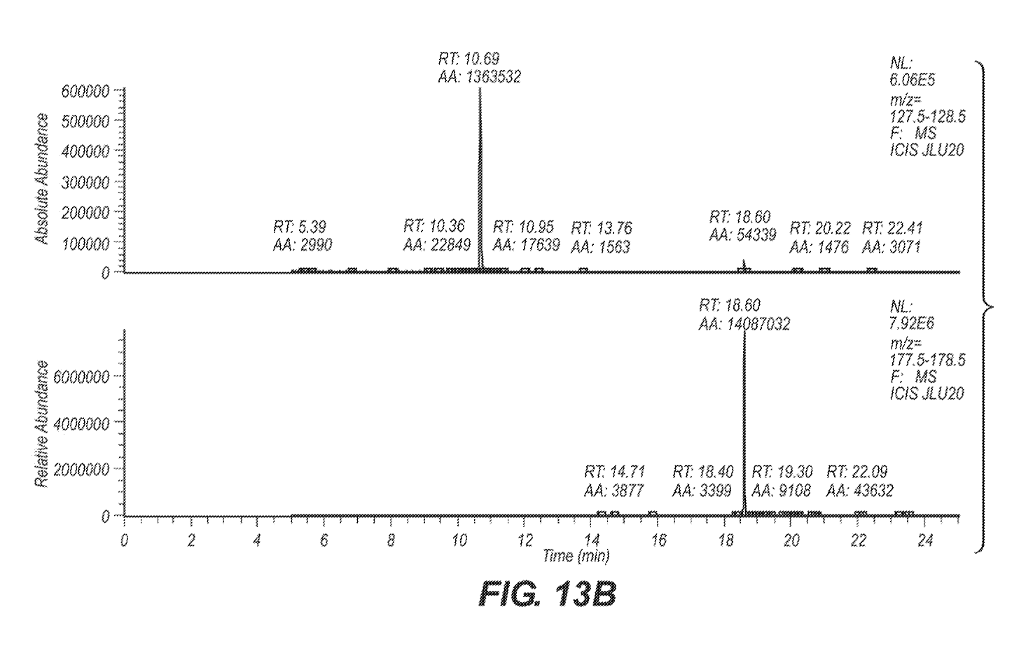

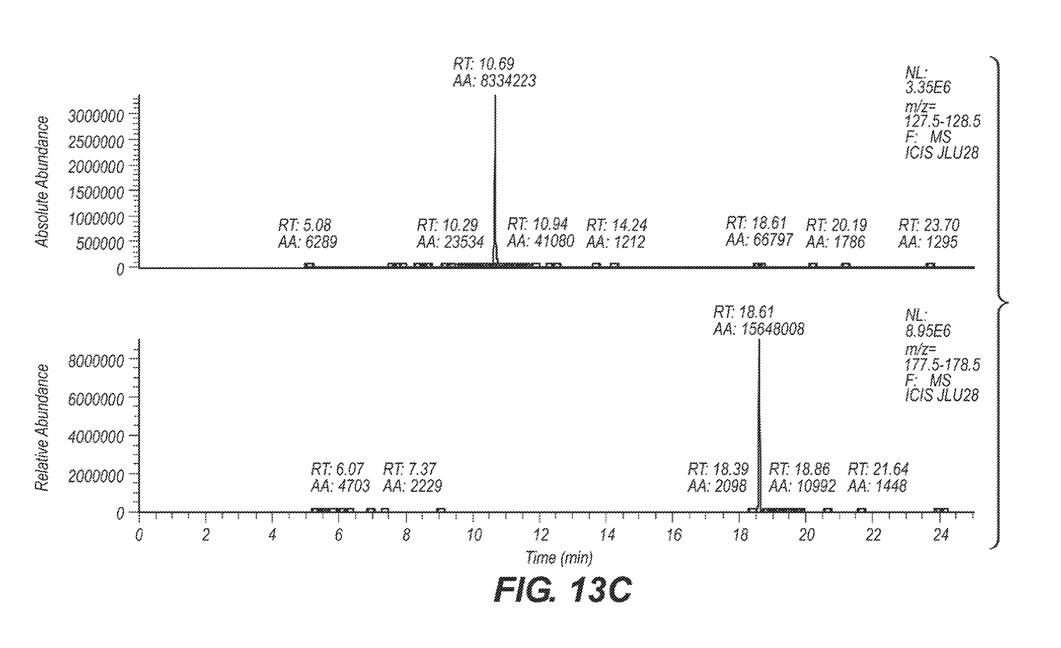

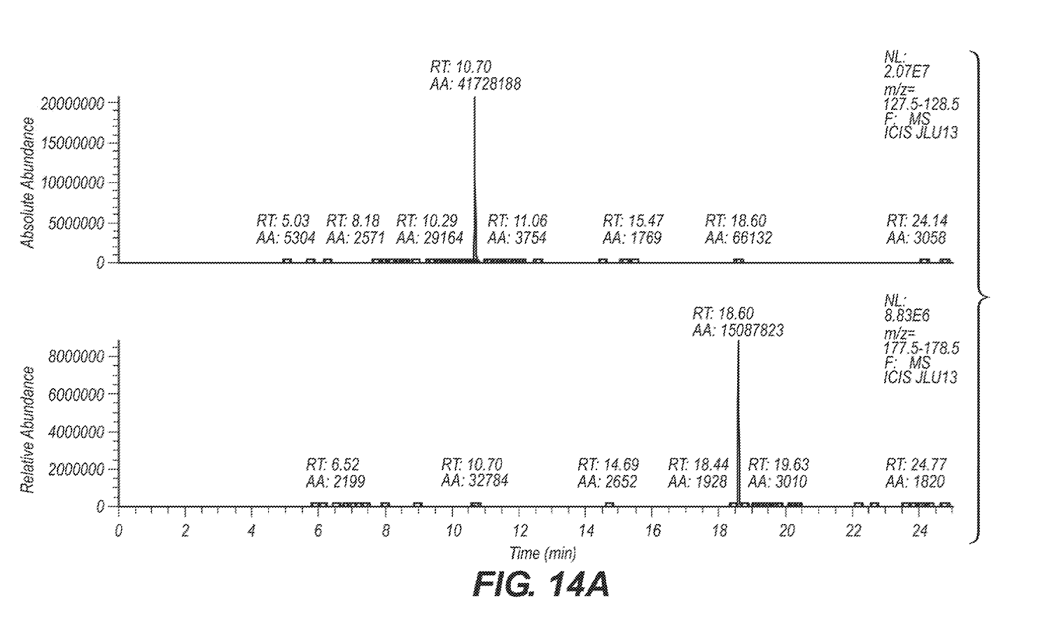

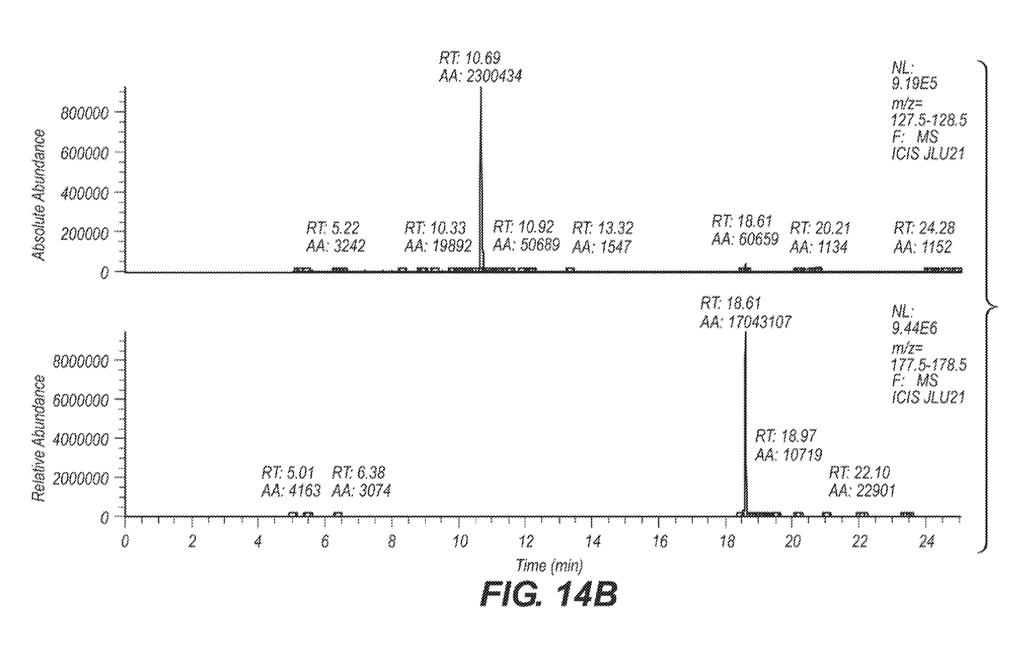

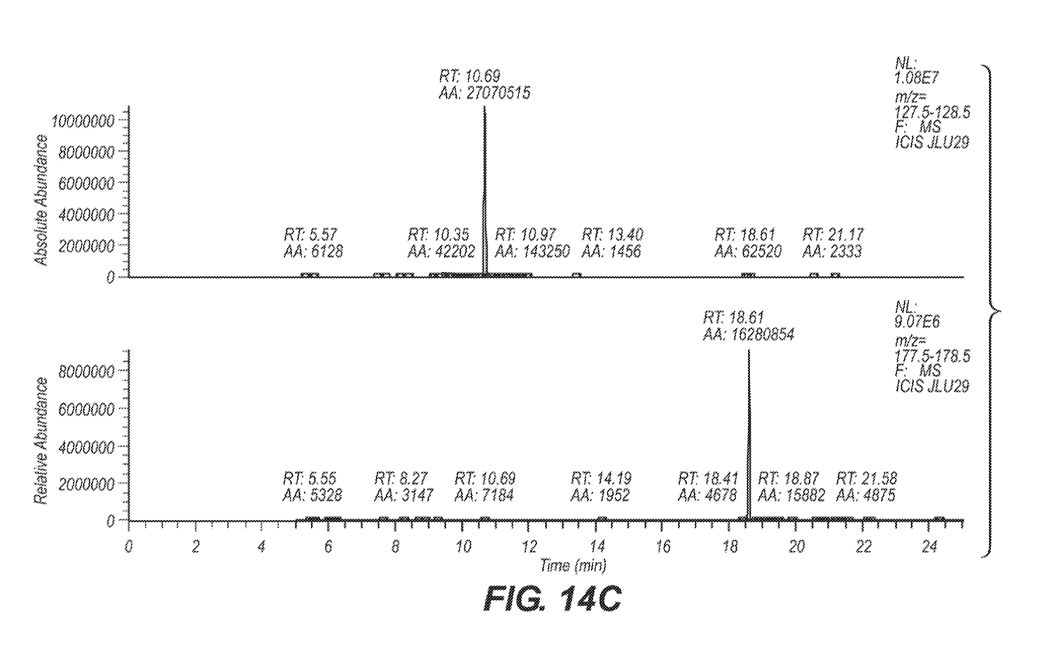

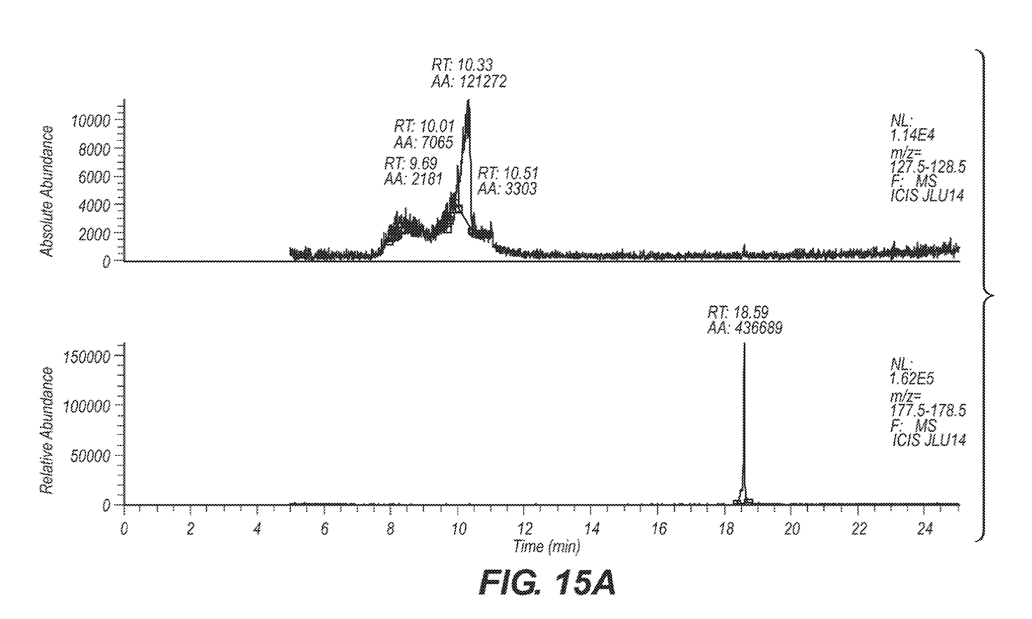

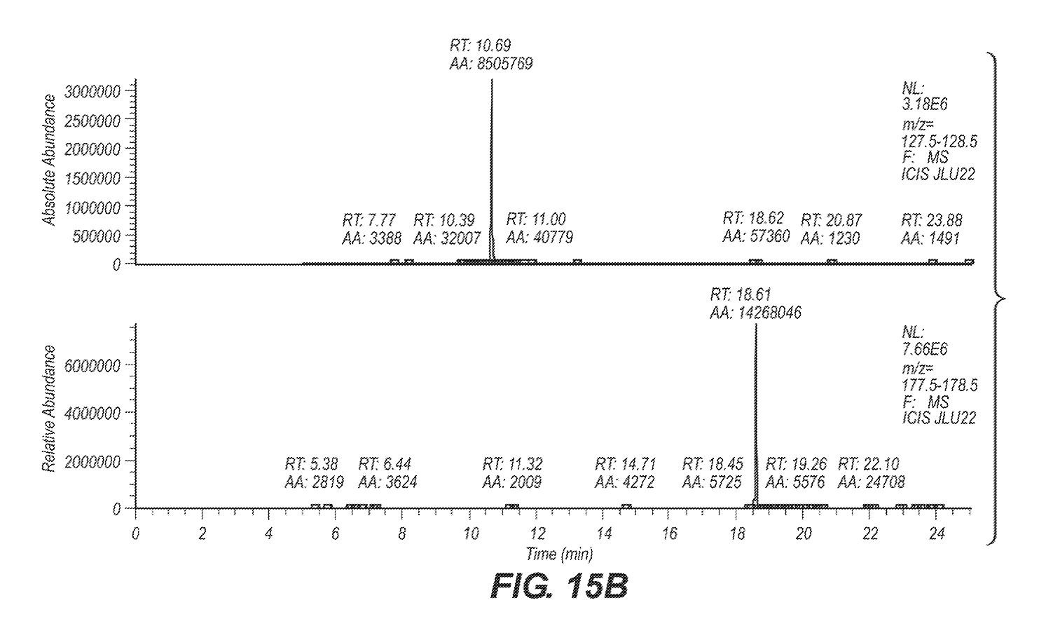

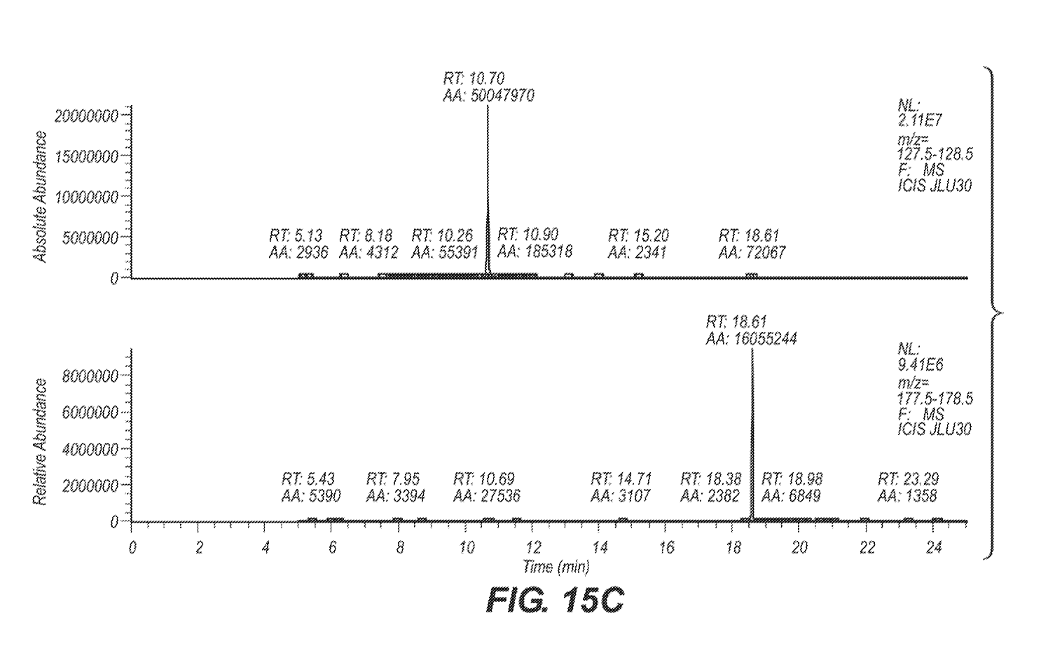

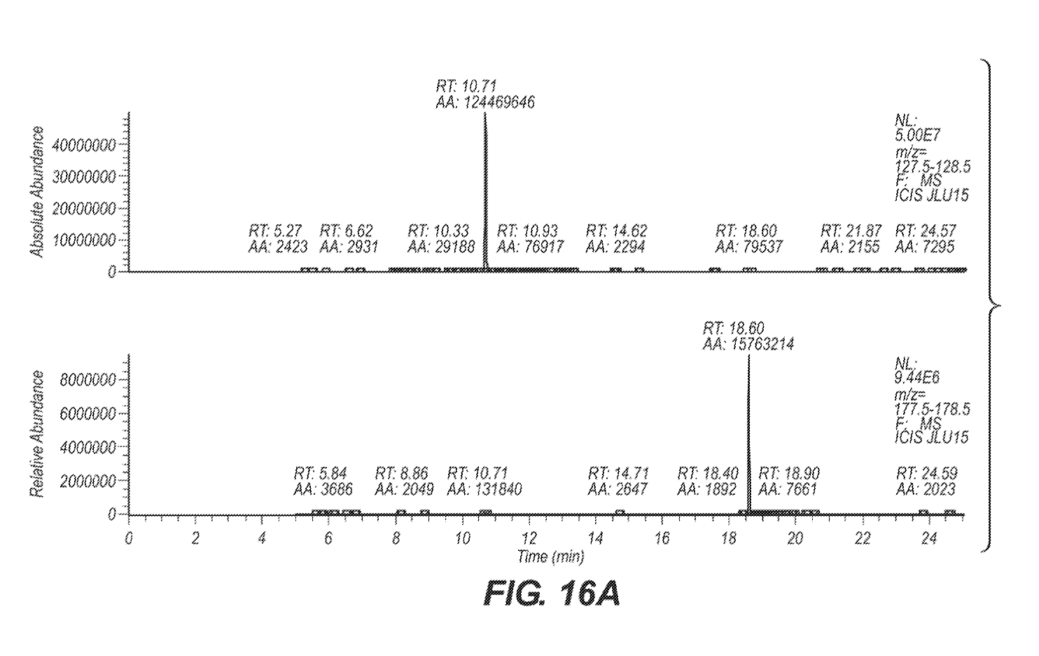

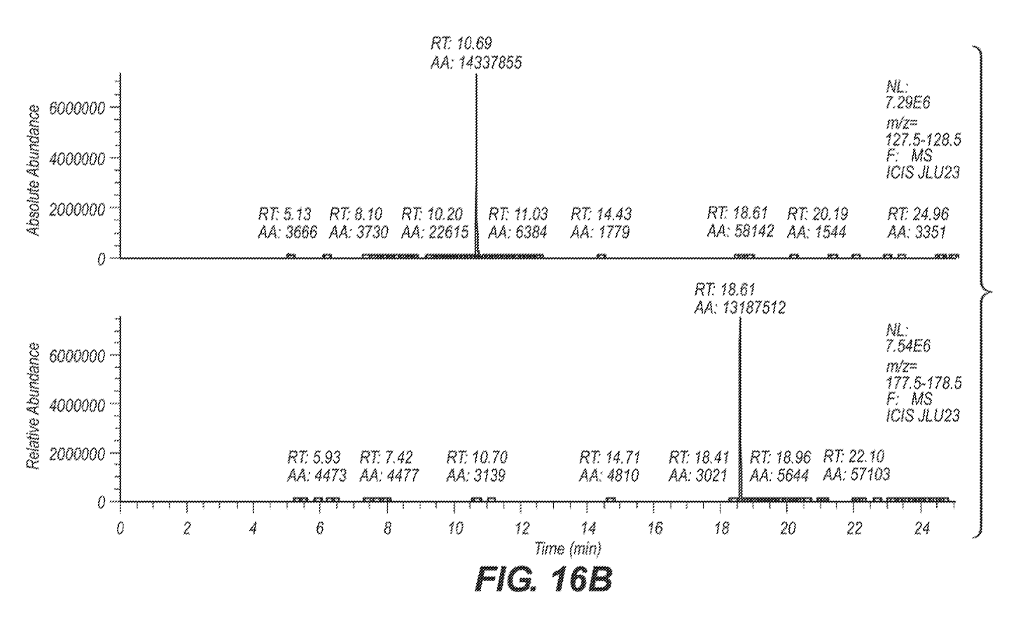

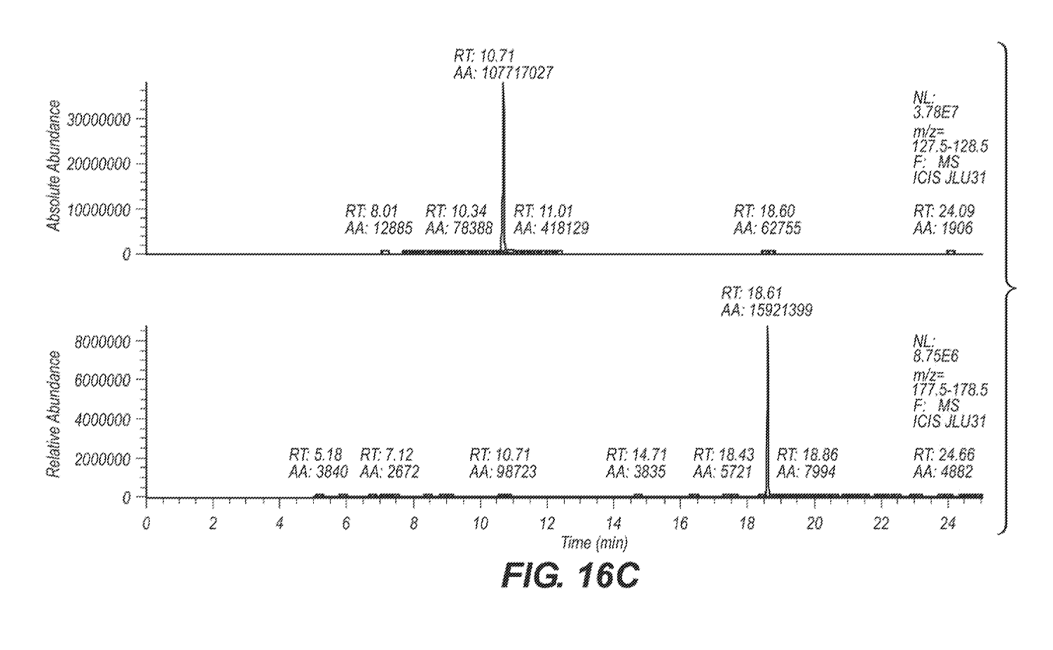

FIGS. 2-8 and 10-16 show the GC/MS chromatographs for various extracts of naphthalene solutions (0.01-20 ppm) with and without polymer treatments. FIGS. 2-8 show results using PHA polymer fibers. FIGS. 10-16 show results using PHA polymer film disks. Each top chromatograph shows the amount of naphthalene extracted without polymer treatment. Each middle chromatographs show the amount of naphthalene left in solution after polymer treatment. The bottom chromatographs show the amount of naphthalene extracted from the polymer. Each of these results is divided into two chromatographs with the top in each section showing the naphthalene concentration, retention time, and m/z ratio identifying the compound as naphthalene. The second chromatograph in each section shows the anthracene internal control.

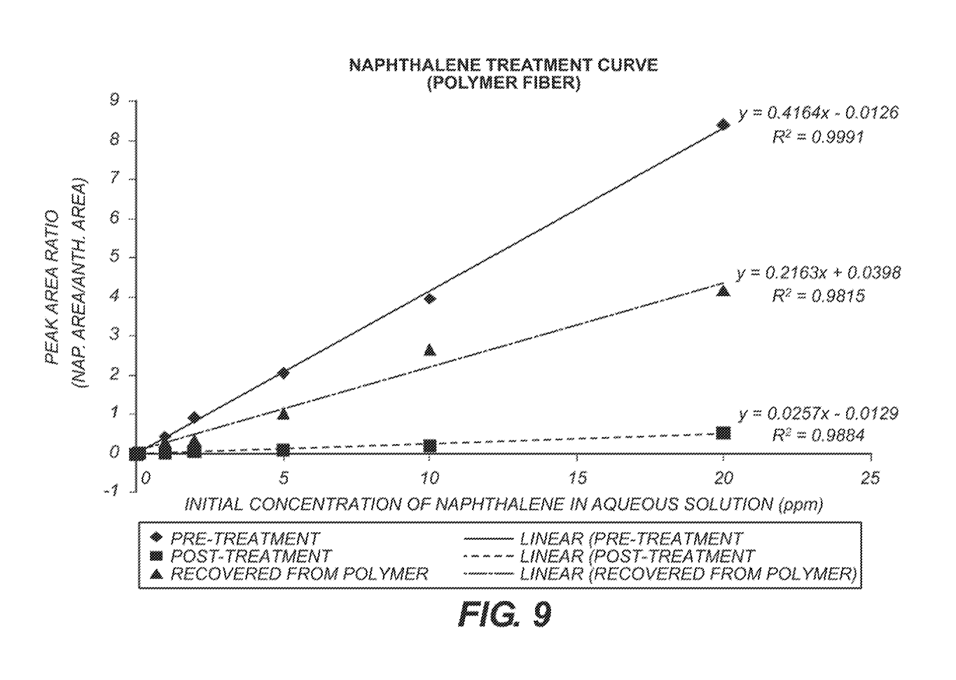

FIG. 9 shows naphthalene treatment curve for PHA polymer fibers. The peak area ratio of naphthalene area to anthracene area is plotted against naphthalene concentration (ppm).

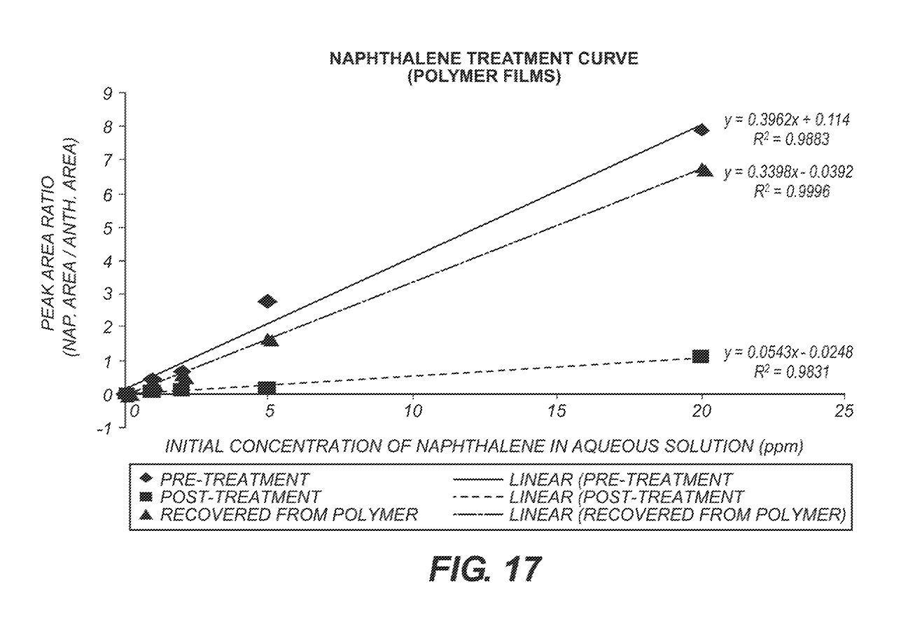

FIG. 17 shows a naphthalene treatment curve for PHA polymer film disks. The peak area ratio of naphthalene area to anthracene area (y-axis) is plotted against naphthalene concentration in ppm (x-axis).

### 6.2 Example 2

### PAH Extraction Using PHA

6.2.1 Introduction

This example demonstrates the extraction of PAH using PHA.

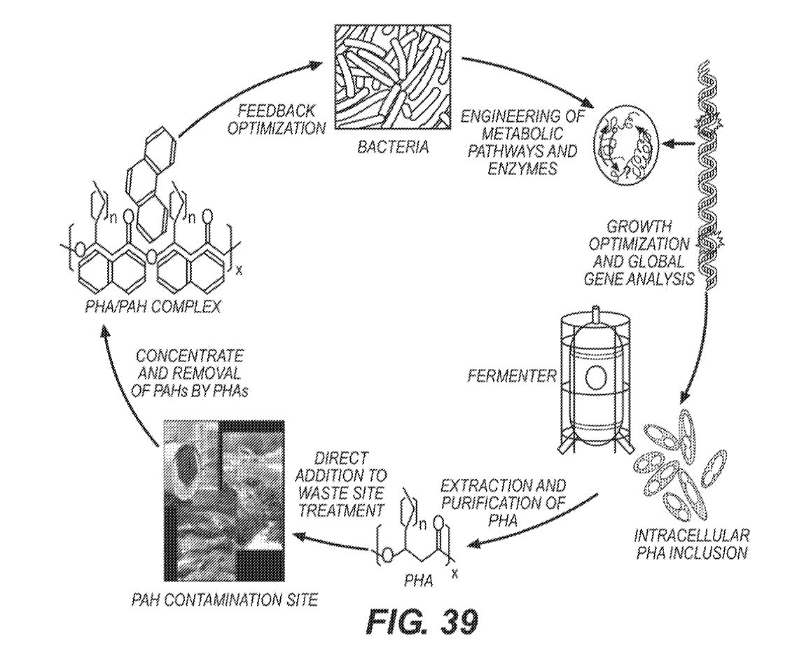

FIG. 39 shows an embodiment of the method for PAH bioremediation using PHA. Biodegradable polymers (e.g., PHAs) can be produced from cells or microbes (e.g., a bacterial strain) using methods known in the art. The cells or microbes (e.g., bacteria) can be genetically engineered using methods well known in the art to express a desired PHA. For example, genetic engineering of a metabolic pathways and/or enzyme can be performed to produce the desired PHA. Growth optimization and global gene analysis can be performed using methods known in the art to determine a microbial strain suitable for production of the desired PHA by fermentation. PHAs can be grown, for example, in a fermenter utilizing carbon sources such as sugar, starch, plant oil, or PAH containing wastes. The PHA can be extracted and purified. The new polymer can be made into commercial useful materials or objects. In one embodiment, the PHA can be used for environmental remediation, e.g., direct addition to waste site treatment. In one embodiment, the PHA can be used to remediate PAH contamination by concentrating and removing the PAHs. The microbial strain can be further selected and/or improved by using additional feedback optimization using methods known in the art and based on the results (e.g., efficiency, effectiveness or extent) of the remediation of PAH contamination.

6.2.2 Materials and Methods

Standards

PAH standards were made using either naphthalene or phenanthrene as a model compound and dissolved in hexanes. The naphthalene standard was prepared at concentrations of 0.01 ppm, 0.1 ppm, 1 ppm, 2 ppm, 5 ppm, 10 ppm, and 20 ppm while standard phenanthrene concentrations were 0.001 ppm, 0.01 ppm, 0.125 ppm, 0.25 ppm, 0.5 ppm, and 1 ppm. Constant concentration of anthracene (2 ppm) was added to the naphthalene standard set as an internal standard. Constant concentration of naphthalene (0.3 ppm) was added to the phenanthrene standard set as an internal standard. All samples were run on Gas Chromatography-Mass Spectrometry (Thermo Finnigan MAT 95XP, US). A DB5 30 m column was used. The column was initially held at 60° C. for 1 min, followed by an increase to 250° C. at a rate of 20° C. per min, finally the column was held at 250° C. for 5 min. The total run time is approximately 16 min. Samples were injected under split-with-surge mode with 0.60 min split time and 220 injection-minute. 2 μl of sample were injected and the flow rate was at 1 ml per min. The mass spectrometry scan parameter was set at 100 amu to 200 amu with 0.44 amu per scan or 25 scans per sec.

Naphthalene and Phenanthrene Samples

Samples of ultra-pure water (10 mL) were spiked with concentrated naphthalene or phenanthrene in methanol. The concentrations of sample tested for naphthalene was 0.01 ppm, 0.1 ppm, 1 ppm, 2 ppm, 5 ppm, 10 ppm, and 20 ppm. The concentrations of sample tested for phenanthrene was 0.001 ppm, 0.01 ppm, 0.125 ppm, 0.25 ppm, 0.5 ppm, and 1 ppm. Naphthalene at 20 ppm and phenanthrene at 1 ppm were the saturation points; however, such concentrations can be found in highly contaminated environmental sites and are readily detected (15). The efficiency of hexanes in extraction of naphthalene or phenanthrene from aqueous solution was ascertained base on the standard curve (pre-treatment).

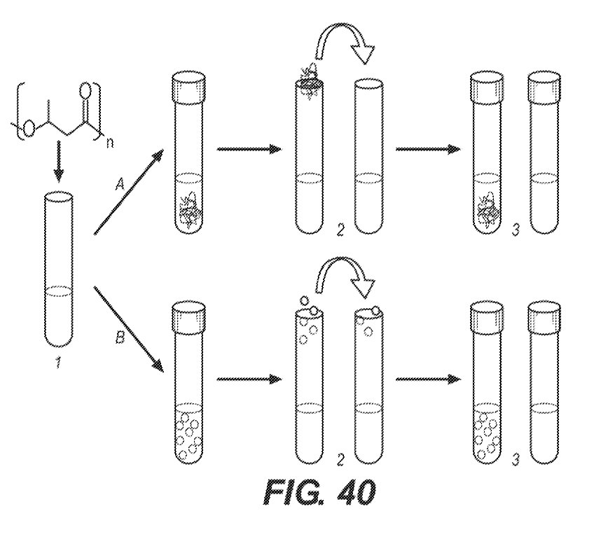

PHA biodegradable polymer fibers or films (30 mg) were added to a set of 10 ml aqueous samples, each with known PAH concentration by immersing the PHA directly in the sample solution. The samples were shaking on a shaker (Taitec Recipro Shaker, Japan) for 6 hours at 160 rpm. The polymer film or fiber was removed from the aqueous solution and extracted with 2 mL hexanes to desorb the bound PAH (recovery). The remaining aqueous solution was also extracted with 2 mL hexanes for comparison with the polymer extricant (post-treatment) (FIG. 40).

FIG. 40 shows the extraction procedure used for the removal of PAHs from aqueous sample. PHA (30 mg) fiber (A) or film (B) was added to each of the 10 ml water with naphthalene concentration of 0.01 ppm, 0.1 ppm, 1 ppm, 2 ppm, 5 ppm, 10 ppm, and 20 ppm or phenanthrene concentration of 0.001 ppm, 0.01 ppm, 0.125 ppm, 0.25 ppm, 0.5 ppm, and 1 ppm (1). After reaching equilibrium (6 h), PHA polymer-naphthalene complexes were taken out and extracted with 2 mL hexanes in another vial to desorb PAH from PHA (2). The remaining aqueous solution after PHA treatment was also extracted with hexanes for comparison with the polymer extricant (3).

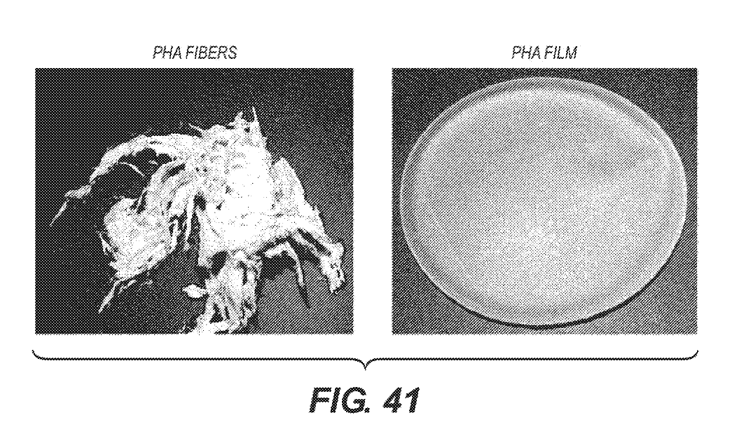

FIG. 41 shows the P[3HB] fibers and film accumulated by E. coli.

Control

Ultra-pure water without the addition of PAH was assayed using the same extraction procedure as described above to insure that there was no PAH contamination in the polymer or ultra-pure water. Since PAHs are extremely hydrophobic, they tend to stick to everything. In order to minimize analyte loss, all glassware and glass pipettes were rinsed with hexanes and counted towards the post-treatment concentration. Each analysis was carried out in triplicate and GC-MS was used to identify and quantify PAH in each sample.

Extraction Rate

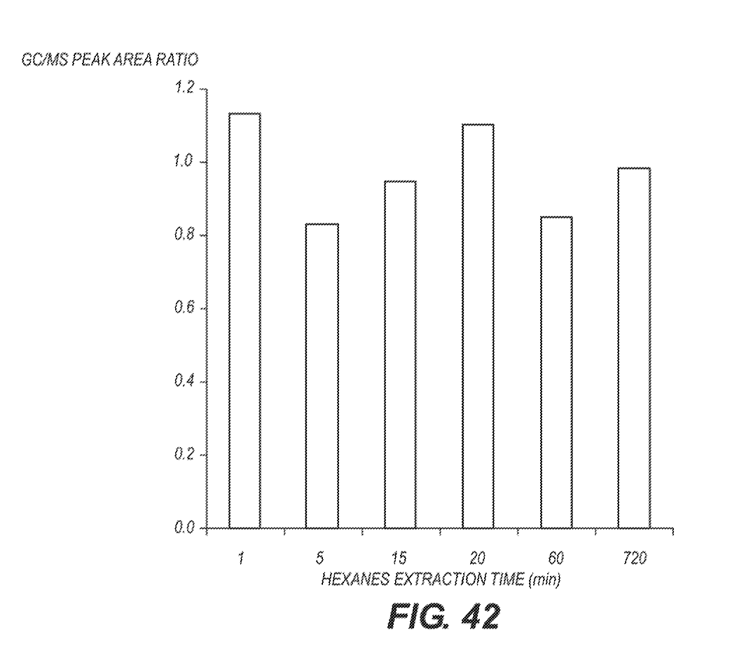

Optimum extraction rate was determined by conducting the same procedure extraction on a set of PAH samples with the same concentration over increasing periods of extraction time. A set of 6 phenanthrene aqueous samples at 0.2 ppm concentration was prepared and extracted with 2 mL of hexanes. The extraction time of each of the 6 samples were varied as follow: 1 min, 5 min, 15 min, 20 min, 60 min, and 720 min (FIG. 42).

FIG. 42 shows phenanthrene (0.2 ppm) extraction using hexanes. Extraction times tested were 1 min, 5 min, 15 min, 20 min, 60 min, and 720 min.

Conditions to Mimic the Environment

The PAH extraction analysis was repeated as described above using phenanthrene as the PAH model compound and carried out using water containing humic substances (40 ppm) to emulate a pseudo-environmental condition.

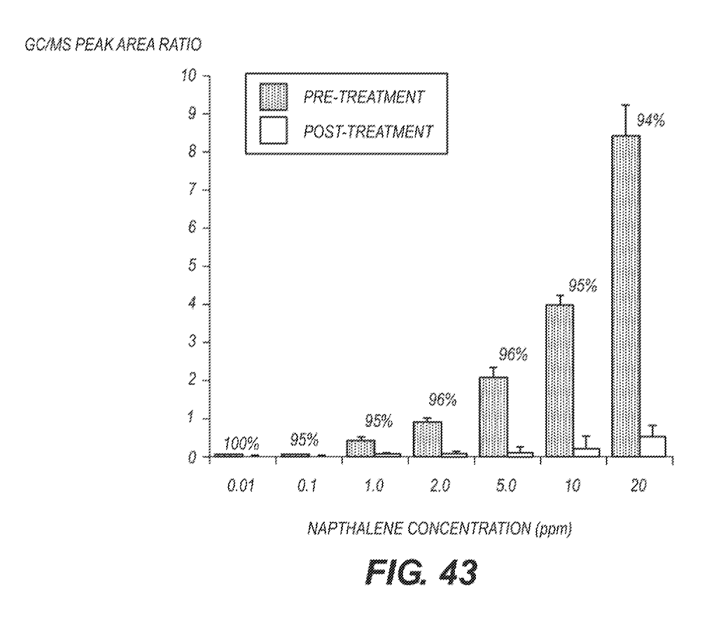

FIG. 43 shows naphthalene sequestration by P[3HB] polymer fiber. The naphthalene concentrations tested were 0.01 ppm, 0.1 ppm, 1 ppm, 2 ppm, 5 ppm, 10 ppm, and 20 ppm. The amount of naphthalene removed was 100%, 95%, 95%, 96%, 96%, 95%, and 94% respectively.

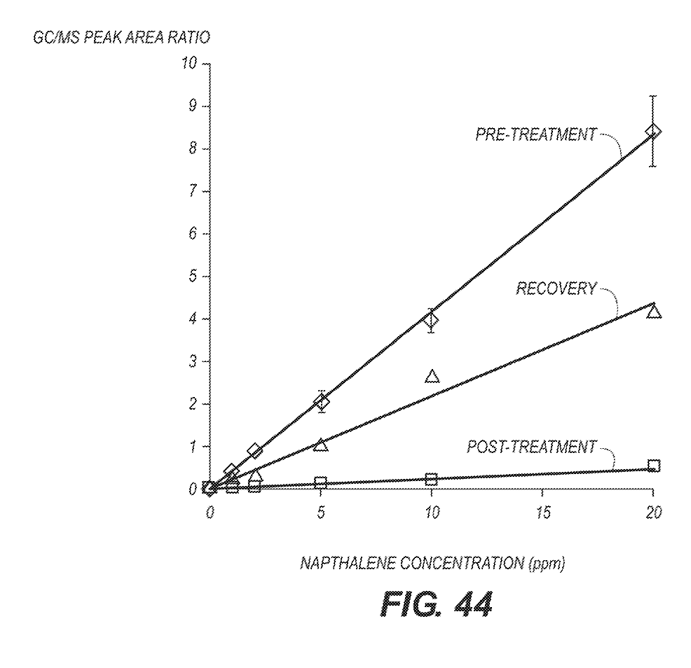

FIG. 44 shows naphthalene removal by P[3HB] polymer fiber.

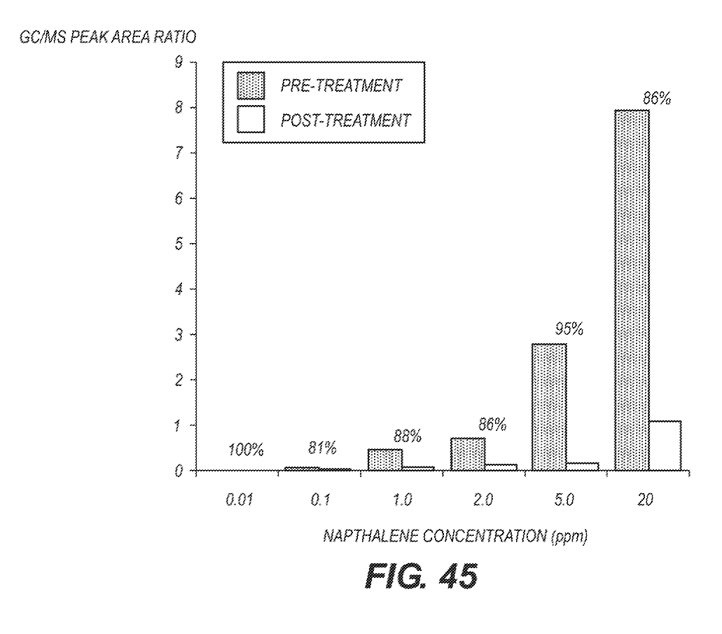

FIG. 45 shows naphthalene sequestration by P[3HB] polymer films. Naphthalene concentrations tested were 0.01 ppm, 0.1 ppm, 1 ppm, 2 ppm, 5 ppm, and 20 ppm. The amount of naphthalene removed was 100%, 81%, 88%, 86%, 95%, and 86% respectively.

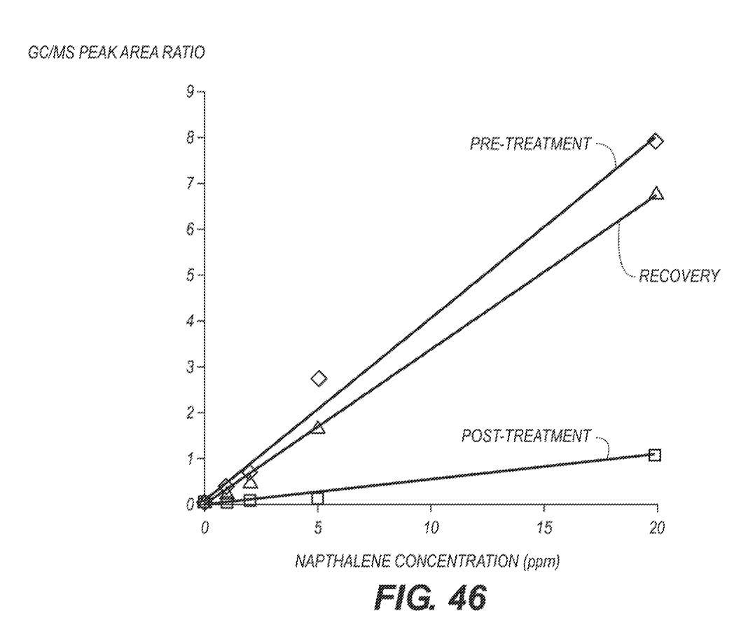

FIG. 46 shows naphthalene removal by P[3HB] polymer films.

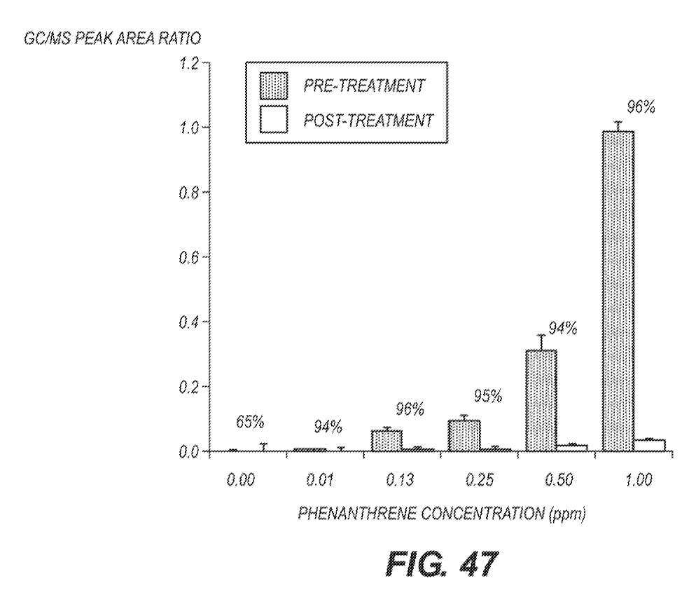

FIG. 47 shows phenanthrene sequestration by P[3HB] polymer fibers. The phenanthrene concentrations tested were 0.001 ppm, 0.01 ppm, 0.125 ppm, 0.25 ppm, 0.5 ppm, and 1 ppm. The amount of phenanthrene removed was 65%, 94%, 96%, 95%, 94%, and 96% respectively.

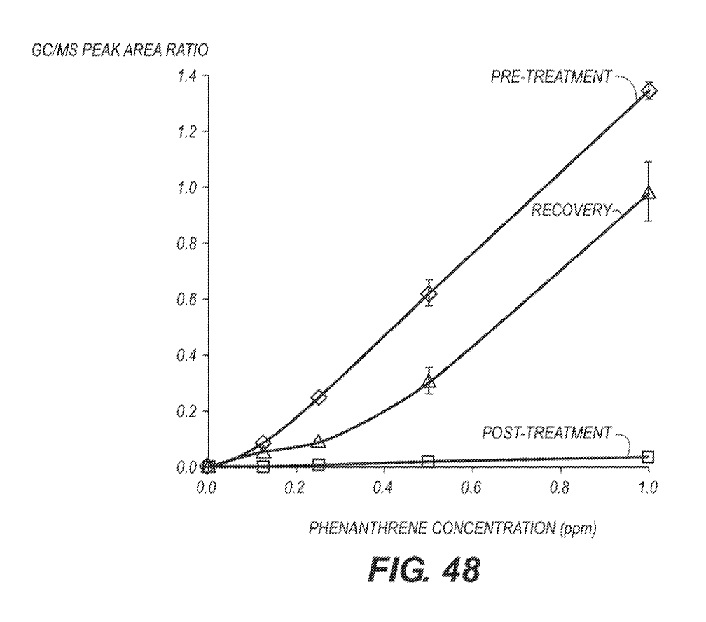

FIG. 48 shows phenanthrene removal by P[3HB] polymer fibers.

### 6.3 Example 3

### Pseudo-Environmental Cleanup of Phenanthrene

6.3.1 Introduction

Extraction of organic pollutants from actual environmental contamination sites is fundamentally different from extraction performed in a laboratory setting. Hydrophobic compounds such as PAHs are strongly absorbed to many organic particles and other soil material. The sorption occurs through a combination of mechanisms including surface adsorption and partitioning or dissolution into organic phases.

This example demonstrates the potential for P[3HB] biodegradable polymers to concentrate and remove PAHs with high efficiency from environmental contamination sites. Humic substances were added to sample solutions to assess whether P[3HB] could competitively remove the PAHs away from the aqueous solution and organic matter. Extraction using a PHA, P[3HB], is extremely efficient and extraction time is 8 times more rapid (6 hours) when compared with the extraction times for other techniques currently in use (48 hours).

One of the major components of humic substances is humic acid. It is commercially available as a dark brown powder. Humic substances are a major part of the natural organic matter in soil and water as well as in geological organic deposits such as lake sediments, peats, brown coals and shales. In this example, 40 ppm humic acid was added to each sample to mimic the settings of PAH cleanup in the environment.

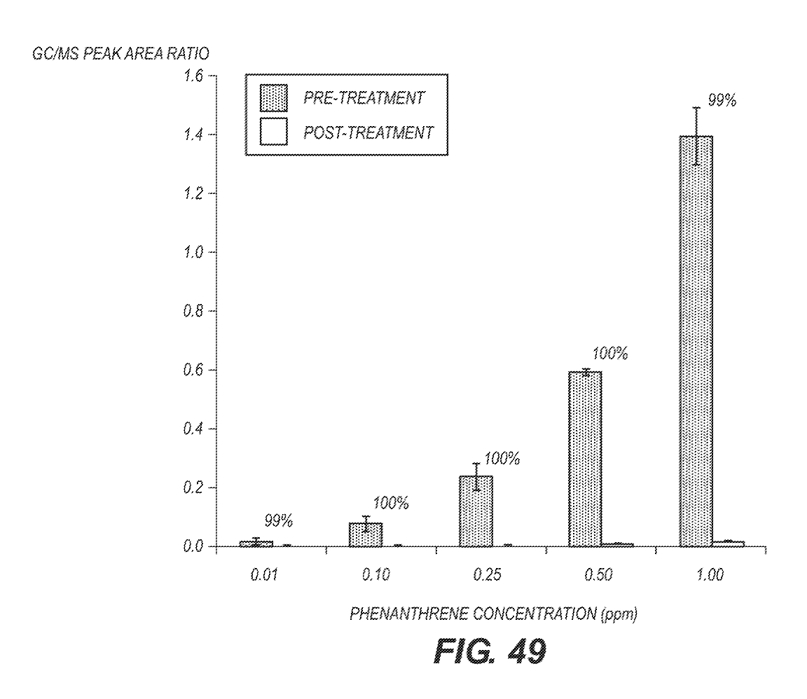

FIG. 49 shows phenanthrene sequestration by P[3HB] polymer fibers in the presence of humic substances. Phenanthrene concentrations of 0.01 ppm, 0.1 ppm, 0.25 ppm, 0.5 ppm, and 1 ppm were tested. The amount of phenanthrene removed was 99%, 100%, 100%, 100%, and 99% respectively.

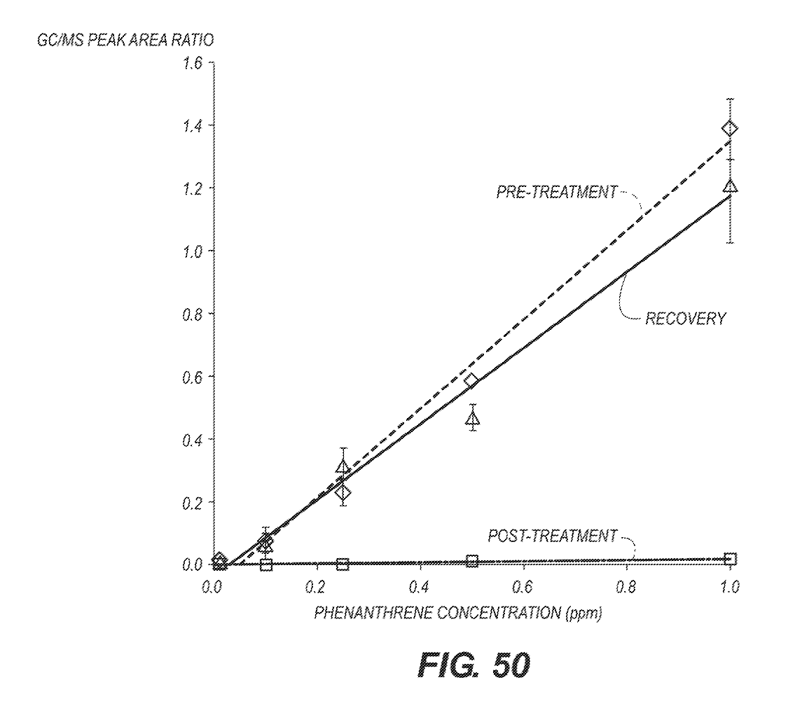

FIG. 50 shows phenanthrene removal by P[3HB] polymer fibers in the presence of humic substances.

Table 1 compares PAH extraction techniques in current use with the PHA extraction technique described in this example. As shown in the table, the PHA extraction time is 8 times more rapid (6 hours) when compared with the extraction times for the other techniques (48 hours).

TABLE 1

| PAH Extraction | | Volume of | |
|---|---|---|---|
| Technique | Solvent Used | Solvent Used | Extraction Time |
| Soxhlet | DCM/acetone | 300 mL/10 mL | 48 hrs |
| | hexanes/acetone | | |
| Sonication | DCM/acetone | 40 mL/10 mL | 48 hrs |
| ASE | isopropanol | 50 mL/10 mL | 48 hrs |
| SFE | dichloromethane | 180 mL/10 mL | 48 hrs |
| | cyclohexane | 250 mL/10 mL | 48 hrs |
| MSE | methanol | 50 mL/10 mL | 48 hrs |
| PHA | hexanes | 2 mL/10 mL | 6 hrs |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A method for adsorbing a hydrophobic organic compound (“HOC”) from a material contaminated with the HOC comprising:
a. providing a polyhydroxyalkanoate (PHA) polymer; and
b. contacting the material contaminated with the HOC with the PHA polymer under conditions wherein the HOC binds with the PHA polymer.

2. A method for sequestering a HOC from a material contaminated with the HOC comprising:
a. loading a PHA polymer that binds with at least one HOC onto a support; and
b. contacting the PHA polymer loaded onto the support to the material contaminated with the HOC.

3. The method of claim 1 further comprising:
c. maintaining environmental conditions to sustain HOC adsorption until a desired amount of adsorption of the HOC is achieved.

4. The method of claim 2 further comprising:
c. maintaining environmental conditions to sustain HOC sequestration until a desired amount of sequestration of the HOC is achieved.

5. The method of claim 1 or 2, wherein the PHA polymer is produced by a microbe.

6. The method of claim 5, wherein the microbe is selected from the group consisting of Escherichia, Pseudomonas, Alcaligenes, Corynebacterium, Acinetobacter, Rhodococcus, Nocardia, Mycobacterium, Bacillus, Flavobacterium, Cunnighamella, Agmenellum, Phanerochaete, Burkholderia, Sphingomonas, Vibrio, Comamonas, and Gordona.

7. The method of claim 1 or 2, wherein the PHA polymer is selected from the group consisting of poly(3-hydroxybutyrate), poly-3-alkanoates (PHA or P(3HA)) such as 3-hydroxypropionate, 3-hydroxyhexanoate, 3-hydroxyoctanoate, 3-hydroxydecanoate, 3-hydroxynonoate, 3-hydroxydodecanoate, 3-hydroxytetradecanoate, 3-hydroxyvalerate, 4-hydroxyalkanoates such as 4-hydroxybutyrate and 4-hydroxyvalerate, 5-hydroxyvalerate, 6-hydroxyhexanoate, phenoxyalkanoates, poly-3-hydroxybutyrate-co-3-hydroxyhexanoate, poly-3-hydroxybutyrate-co-3-hydroxyoctanoate, poly-3-hydroxybutyrate-co-3-hydroxydecanoate, poly-4-hydroxybutyrate, poly-4-hydroxybutyrate-co-3-hydroxybutyrate, poly-4-hydroxybutyrate-co-2-hydroxybutyrate, and copolymers and blends thereof.

8. The method of claim 1 or 2, wherein the PHA polymer is a bacterial biopolythioester.

9. The method of claim 1 or 2, wherein the material contaminated with the HOC is a gas, gaseous fluid, fluid or solid.

10. The method of claim 1 or 2, wherein the PHA polymer comprises one or more units of formula 1: —OCR¹R²(CR³R⁴)ₙCO—;
wherein n is an integer and wherein R^{1,} R^{2,} R³ and R⁴ independently are hydrocarbon radicals.

11. The method of claim 10, wherein the PHA polymer comprises between 10 and 100,000 units of formula 1.

12. The method of claim 10, wherein the PHA polymer comprises between 100 and 30,000 units of formula 1.

13. The method of claim 10 wherein n is an integer between 1 and 15.

14. The method of claim 10 wherein the hydrocarbon radicals are selected from the group consisting of long chain hydrocarbon radicals, halo- and hydroxy-substituted radicals, hydroxy radicals, halogen radicals, nitrogen-substituted radicals, oxygen-substituted radicals, and/or hydrogen atoms.

15. The method of claim 1 or 2, wherein the PHA polymer is a fiber.

16. The method of claim 1 or 2, wherein the PHA polymer is a film.

17. A method for remediating a material contaminated with a HOC comprising:
a. providing a PHA polymer;
b. contacting the material contaminated with the HOC with the PHA polymer under conditions wherein the HOC binds with the PHA to form a PHA polymer-HOC complex; and
c. removing the HOC from the PHA polymer-HOC complex.

18. The method of claim 17 wherein the material is a gas, gaseous fluid, fluid or solid.

19. The method of claim 17 additionally comprising:
d. providing a microbe that catabolizes or sequesters the PHA polymer, wherein the step of removing the HOC from the PHA polymer-HOC complex comprises catabolism or sequestration by the microbe.

20. The method of claim 19 wherein the microbe is selected from the group consisting of Escherichia, Pseudomonas, Alcaligenes; Corynebacterium, Acinetobacter, Rhodococcus, Nocardia, Mycobacterium, Bacillus, Flavobacterium, Cunnighamella, Agmenellum, Phanerochaete, Burkholderia, Sphingomonas, Vibrio, Comamonas, and Gordona.

21. A method for detecting a HOC in a material comprising:
a. providing a PHA polymer;
b. contacting the material with the PHA polymer under conditions wherein the HOC binds with the PHA polymer; and
c. assaying for the presence of HOC bound with the PHA polymer.

22. The method of claim 21 wherein the material is a gas, gaseous fluid, fluid or solid.

23. The method of claim 21 wherein the assaying step comprises determining a concentration of the HOC bound with the PHA polymer.

24. The method of claim 21 wherein the assaying step comprises performing gas chromatography and mass spectroscopy (GC/MS).

25. The method of claim 21 wherein the assaying step comprises performing HPLC, HPLC/MS, or NMR.

26. An apparatus for adsorbing a HOC from a material contaminated with the HOC comprising:
a. a PHA polymer that binds with at least one HOC,
wherein the PHA polymer is disposed in the apparatus in an orientation so that it contacts the material contaminated with the HOC.

27. The apparatus of claim 26 wherein the PHA polymer is loaded on a support.

28. The apparatus of claim 26 wherein the PHA polymer is a fiber.

29. The apparatus of claim 26 wherein the PHA polymer is a film.

30. The apparatus of claim 26 that maintains environmental conditions to sustain HOC adsorption or sequestration until a desired amount of adsorption or sequestration of the HOC is achieved.

31. The apparatus of claim 26 which is a filter, wand, cone or tube.

32. The apparatus of claim 26 which is a coating.

33. The apparatus of claim 27 wherein the support is selected from the group consisting of metal rod, metal tube, metal mesh, glass rod, glass tube or glass mesh.

34. The apparatus of claim 27 wherein the support is a gelatinous medium.

35. The apparatus of claim 27 wherein the support is selected from the group consisting of gels, pastes, paper, and aqueous solutions.

36. The apparatus of claim 35 wherein the support is a filter.

37. An apparatus for detecting a HOC in a material comprising:
a PHA polymer that binds with at least one HOC, wherein:
the PHA polymer is disposed in the apparatus in an orientation so that it contacts the material;
the PHA polymer binds with the HOC in the material; and
the presence of the HOC in the PHA polymer is determined by performing an assay for the HOC.

38. The apparatus of claim 37 wherein the assay comprises determining a concentration of the HOC bound with the PHA polymer.

39. The apparatus of claim 37 wherein the assay comprises performing gas chromatography and mass spectroscopy (GC/MS).

40. A method for producing a PHA selected from the group consisting of the synthetic reaction pathways (#18-1-18-15) as set forth in FIG. 18 and combinations thereof.

41. A method for producing a PHA selected from the group consisting of the synthetic reaction pathways (#27-1-27-6) as set forth in FIG. 27 and combinations thereof.

42. The method of claim 41 selected from the group consisting of the synthetic reaction pathways set forth in FIGS. 29-36 and 38.
